(19) United States
(12) Patent Application Publication
(10) Pub. No.: US 2025/0334554 A1
(43) Pub. Date: Oct. 30, 2025

(54) ANALYTICAL METHOD FOR QUANTIFYING SUCRALOSE-6-ACETATE CONTENT

(71) Applicant: Avazyme, Inc., Oxford, NC (US)

(72) Inventors: Stephen Werness, Durham, NC (US); Volker Bornemann, Durham, NC (US)

(21) Appl. No.: 19/241,241

(22) Filed: Jun. 17, 2025

Related U.S. Application Data
(60) Provisional application No. 63/660,995, filed on Jun. 17, 2024.

(51) Int. Cl. G01N 30/72; G01N 30/02; G01N 30/04; G01N 30/86; G01N 30/88

(52) U.S. Cl. CPC G01N 30/7233; G01N 30/8631; G01N 30/88; G01N 2030/027; G01N 2030/045; G01N 2030/8836

(57) ABSTRACT

Sucralose-6-acetate is the chemical precursor the artificial sweetener sucralose. The safety of sucralose-6-acetate has come into question after recent studies have demonstrated small amounts may be genotoxic (0.15 μg/day). To date, no analytical methods exist that can quantitatively determine sucralose-6-acetate content in sucralose at levels sufficient to ensure consumer safety upon sucralose consumption. To this end, the present inventors have developed novel analytical methods that utilize liquid chromatography coupled with mass spectrometry that can quantitatively determining amounts of sucralose-6-acetate present in commercially available solid- and liquid-sucralose samples at parts per billion levels.

### CROSS-REFERENCE TO RELATED APPLICATIONS

This application claims the benefit of U.S. Provisional Application No. 63/660,995 filed Jun. 17, 2024, the contents of which are hereby incorporated by reference in their entirety.

### BACKGROUND

As of 2019, roughly 37.3 million Americans, or approximately 11.3% of the population, have diabetes. Among diabetes diagnoses, the overwhelming majority are Type 2, defining roughly 95% of cases. In Type 2 diabetes, there are primarily two problems: (i) the pancreas does not produce enough insulin (a hormone that regulates the movement of sugar into the cells); and (ii) cells respond poorly to insulin. There is currently no cure for Type 2 diabetes, however, those with Type 2 diabetes can be prescribed medicines, exercise, and eat a healthy diet to help manage the disease. In addition to these management strategies, people with Type 2 diabetes will often seek out food products that are sugar-free to help manage their blood sugar.

To this end, numerous artificial sweeteners have been developed over the last 50 years as sugar substitutes. At present, the global market for artificial sweeteners is approximately $7.2 bn (USD) and is expected to grow to $9.7 bn (USD) by 2028.

In the United States, the first artificial sweetener—saccharin, sold under the brand names Sweet and Low®, Sweet Twin®, Sweet'N Low®, and Necta Sweet®—was approved by the FDA in 1977. Since the approval of saccharin, the FDA has approved five additional artificial sweeteners as food additives—they are: (i) aspartame (sold under the brand names Nutrasweet®, Equal®, and Sugar Twin®); (ii) acesulfame potassium (Ace-K) (sold under the brand names Sunett® and Sweet One®); (iii) sucralose (sold under the brand name Splenda®); (iv) neotame (sold under the brand name Newtame®); and (v) advantame, which received general-purpose approval, under certain conditions of use, in 2014. Among these FDA approved sweeteners, aspartame (sold as Nutrasweet®, Equal®, and Sugar Twin®) and sucralose (sold as Splenda®) command the largest segment of the global artificial sweetener market.

Recently, the safety profiles of artificial sweeteners—particularly, aspartame and sucralose—have been called into question. For example, in 2023, the International Agency for Research on Cancer, an intergovernmental agency forming part of the World Health Organization, classified aspartame as possibly carcinogenic to humans (Group 2B). See Riboli et al., “Carcinogenicity of aspartame, methyleugenol, and isoeugenol,” Lancet Oncol., 2023, 24 (8), 848-850. Likewise, the safety of sucralose has been called into question by numerous studies. For example, studies have demonstrated that: (i) sucralose may be harmful to the gut microbiome (see Schiffman et al., “Sucralose, A Synthetic Organochlorine Sweetener: Overview Of Biological Issues,” J. Toxicol. Environ. Health B, 2013, 16 (7), 399-451; see also Bian et al., “Gut Microbiome Response to Sucralose and Its Potential Role in Inducing Liver Inflammation in Mice,” Front. Physiol., 2017, 8 (487), 1-13; see also Chi et al., “Chronic sucralose consumption inhibits farnesoid X receptor signaling and perturbs lipid and cholesterol homeostasis in the mouse livers, potentially by altering gut microbiota functions,” Sci. Total Environ., 2024, 919, 169603); (ii) sucralose may promote obesity (see Yang, “Gain Weight by “Going Diet?” Artificial Sweeteners and the Neurobiology of Sugar Cravings,” Yale J. Biol. Med., 2010, 83 (2), 101-108; see also Wang et al., “Sucralose Promotes Food Intake through NPY and a Neuronal Fasting Response,” Cell Metab., 2016, 24 (1), 75-90); (iii) sucralose can damage cells of the pancreas (see Gupta et al., “Sucralose induced pancreatic toxicity in albino rats: Histomorphological evidence,” J. Morphol. Sci., 2014, 31 (2), 123-127); and (iv) sucralose can cause insulin resistance (see Mathur et al., “Effect of artificial sweeteners on insulin resistance among type-2 diabetes mellitus patients,” J. Family Med. Prim. Care, 2020, 9 (1), 69-71; see also Yanina, “The not-so-sweet effects of sucralose on blood sugar control,” Am. J. Clin. Nutr., 2018, 108 (3), 431-432).

The synthesis of sucralose has been known for nearly 30 years. In some synthetic schemes, such as Hao (U.S. Pat. No. 7,932,380), the synthesis of sucralose requires three steps: (i) reacting sucrose with a chlorinating agent, e.g., thionyl chloride, to make chlorinated sucrose; (ii) reacting chlorinated sucrose with an acylating/acetylating agent, e.g., sodium acetate, to make sucralose-6-acetate; and (iii) deacetylating sucralose-6-acetate with a base, e.g., sodium methoxide, to make sucralose. In other synthetic schemes, such as Micinski et al. (U.S. Pat. No. 8,921,540), the synthesis of sucralose also requires three steps: (i) reacting sucrose with an acylating/acetylating agent, e.g., acetic anhydride, to make sucrose-6-acetate; (ii) reacting sucrose-6-acetate with a chlorinating agent, e.g., a chloroformiminium salt, to make sucralose-6-acetate; and (iii) deacetylating sucralose-6-acetate with a base, e.g. sodium methoxide, to make sucralose. While various methods for synthesizing sucralose may perform reaction steps in different orders, the vast majority of methods for making sucralose share the common reaction step of converting sucralose-6-acetate to sucralose by hydrolysis/deacetylation.

Notably, studies have also demonstrated that sucralose-6-acetate, like sucralose itself, may pose safety concerns for consumers. In 2023, for example, one study found that sucralose-6-acetate: (i) may be genotoxic at extremely low consumption levels (0.15 μg/day); (ii) may increase inflammation and oxidative stress; and (iii) may cause cancer in human intestinal cells. See Schiffman et al., “Toxicological and pharmacokinetic properties of sucralose-6-acetate and its parent sucralose: in vitro screening assays,” J. Toxicol. Environ. Health B, 2023, 26 (6), 307-341.

The conventional analytical methods that are currently used to characterize sucralose include: (i) high-performance liquid chromatography (HPLC) with ultraviolet (UV) detection; (ii) HPLC with refractive index (RI) detection; and (iii) thin layer chromatography (TLC). These conventional analytical methods, however, lack sufficient sensitivity and/or selectivity to accurately quantify the sucralose-6-acetate content of sucralose at the ppm or ppb levels required to ensure that sucralose does not contain potentially genotoxic levels of sucralose-6-acetate (i.e., 0.15 μg). The United States Pharmacopeia (USP) monograph of sucralose, for example, identifies two of the aforementioned methods: (i) HPLC with RI detection for sucralose identification; and (ii) TLC for related compound detection. The TLC method specified in the USP monograph of sucralose determines the content of related compounds, such as sucralose-6-acetate, in a 100 mg/mL solution with an acceptance criteria of less than 0.5% (0.5 mg/mL). In other words, the USP monograph of sucralose permits sucralose to contain up to 0.5% of a sucralose-related compound, such as sucralose-6-acetate. At this concentration, sucralose can contain up to 0.5 mg/100 mg of sucralose-6-acetate, which is 5 mg/g or 5,000 ppm (relative to sucralose). Consistent with the USP monograph of sucralose, specification sheets for sucralose from prominent commercial manufacturers of sucralose, e.g., Tate & Lyle, use the same analytical method for sucralose-related compounds (TLC) and specify the same acceptance criteria of 0.5%, or 5,000 ppm. See Tate & Lyle Splenda® Sucralose—Micronized Specification Sheet; see also Tate & Lyle Splenda® Sucralose-Granular (DFF-1) Specification Sheet.

Because sucralose-6-acetate is the synthetic precursor to sucralose and a small amount of sucralose-6-acetate may be genotoxic (i.e., 0.15 μg/day), it is critically important that the analytical methods used to analyze sucralose, and the products that contain it, can accurately quantify sucralose-6-acetate content at exceeding low concentrations. Indeed, the importance of having sensitive analytical methods to quantify sucralose-6-acetate content is clear when considering consumer products:
1. An average energy drink sweetened with sucralose contains approximately 24 mg of sucralose per liter, or 8.52 mg per 12 oz/355 mL drink. See German Federal Institute for Risk Assessment, Opinion No. 006/2023. If a consumer were to consume one average energy drink sweetened with sucralose per day, to ensure that the consumer were not ingesting a potentially genotoxic concentration of sucralose-6-acetate (i.e., 0.15 μg/day), an analytical method would need to detect sucralose-6-acetate at a concentration of: (i) 0.423 ppb (if the sucralose-6-acetate content of the finished energy drink product were analyzed); or (ii) 17.65 ppm (if the sucralose-6-acetate content of the sucralose used to make the energy drink product were analyzed).
2. An average cola sweetened with sucralose contains approximately 110 mg of sucralose per liter, or 39.1 mg per 12 oz/355 mL drink. See German Federal Institute for Risk Assessment, Opinion No. 006/2023. If a consumer were to consume one average cola sweetened with sucralose per day, to ensure that the consumer were not ingesting a potentially genotoxic concentration of sucralose-6-acetate (i.e., 0.15 μg/day), an analytical method would need to detect sucralose-6-acetate at a concentration of: (i) 0.423 ppb (if the sucralose-6-acetate content of the finished cola product were analyzed); or (ii) 3.84 ppm (if the sucralose-6-acetate content of the sucralose used to make the cola product were analyzed).
3. An average flavored water sweetened with sucralose contains approximately 115.7 mg of sucralose per liter, or 41.1 mg per 12 oz/355 mL drink. See DPO International, “What is the level of sucralose is typically used in flavoured water?” 2015. If a consumer were to consume one average flavored water sweetened with sucralose per day, to ensure that the consumer were not ingesting a potentially genotoxic concentration of sucralose-6-acetate (i.e., 0.15 μg/day), an analytical method would need to detect sucralose-6-acetate at a concentration of: (i) 0.423 ppb (if the sucralose-6-acetate content of the finished flavored water product were analyzed); or (ii) 3.65 ppm (if the sucralose-6-acetate content of the sucralose used to make the flavored water product were analyzed).
4. A single Splenda® packet (1 g) contains 1.10% sucralose, or 11 mg per packet. See Abou-Donia et al., “Splenda alters gut microflora and increases intestinal p-glycoprotein and cytochrome p-450 in male rats,” J. Toxicol. Environ. Health A, 2008, 71 (21), 1415-1429. If a consumer were to consume one Splenda® packet per day, to ensure that the consumer were not ingesting a potentially genotoxic concentration of sucralose-6-acetate (i.e., 0.15 μg/day), an analytical method would need to detect sucralose-6-acetate at a concentration of: (i) 150 ppb (if the sucralose-6-acetate content of the Splenda® packet were analyzed); or (ii) 13.63 ppm (if the sucralose-6-acetate content of the sucralose used to make Splenda® were analyzed).

Notably, the foregoing concentrations are orders of magnitude lower than the concentrations that can be quantitatively determined using the existing analytical methods in this field to determine sucralose-6-acetate content, i.e., 5,000 ppm. As such, the existing analytical methods in this field for quantifying sucralose-6-acetate content lack the ability to do so at the relevant levels to ensure that a potentially genotoxic level of sucralose-6-acetate is not present in sucralose, or the products that contain it.

To address this long unmet need, the inventors have developed novel analytical methods that are sufficiently sensitive and selective to accurately quantify the content of sucralose-6-acetate in sucralose-containing materials at the levels necessary to ensure that a potentially genotoxic level of sucralose-6-acetate is not present in sucralose, or the products that contain it. The analytical methods of the present invention are liquid chromatography (LC)-based analytical methods that use mass spectrometry (MS) or tandem mass spectrometry (MS/MS) to quantitatively determine sucralose-6-acetate content in various sucralose-containing mediums at ppb (ng/g) concentrations. These analytical methods are an essential tool that can, and should, be used to ensure that sucralose-containing products do not contain potentially genotoxic levels of sucralose-6-acetate and are therefore safe for consumption.

### SUMMARY

Embodiments of the present disclosure relate to high-performance liquid chromatography (HPLC)-based analytical methods that use mass spectrometry (MS) to quantitatively determine sucralose-6-acetate content in various sucralose-containing mediums.

Embodiments of the present disclosure relate to HPLC-based analytical methods that use tandem mass spectrometry (MS/MS) to quantitatively determine sucralose-6-acetate content in various sucralose-containing mediums.

Embodiments of the present disclosure relate to ultra-high-performance liquid chromatography (UHPLC)-based analytical methods that use MS to quantitatively determine sucralose-6-acetate content in various sucralose-containing mediums.

Embodiments of the present disclosure relate to UHPLC-based analytical methods that use MS/MS to quantitatively determine sucralose-6-acetate content in various sucralose-containing mediums.

Embodiments of the present disclosure relate to isocratic-based methods for use in HPLC-based analytical methods that use MS to quantitatively determine sucralose-6-acetate content in various sucralose-containing mediums.

Embodiments of the present disclosure relate to isocratic-based methods for use in HPLC-based analytical methods that use MS/MS to quantitatively determine sucralose-6-acetate content in various sucralose-containing mediums.

Embodiments of the present disclosure relate to isocratic-based methods for use in UHPLC-based analytical methods that use MS to quantitatively determine sucralose-6-acetate content in various sucralose-containing mediums.

Embodiments of the present disclosure relate to isocratic-based methods for use in UHPLC-based analytical methods that use MS/MS to quantitatively determine sucralose-6-acetate content in various sucralose-containing mediums.

Embodiments of the present disclosure relate to gradient-based methods for use in HPLC-based analytical methods that use MS to quantitatively determine sucralose-6-acetate content in various sucralose-containing mediums.

Embodiments of the present disclosure relate to gradient-based methods for use in HPLC-based analytical methods that use MS/MS to quantitatively determine sucralose-6-acetate content in various sucralose-containing mediums.

Embodiments of the present disclosure relate to gradient-based methods for use in UHPLC-based analytical methods that use MS to quantitatively determine sucralose-6-acetate content in various sucralose-containing mediums.

Embodiments of the present disclosure relate to gradient-based methods for use in UHPLC-based analytical methods that use MS/MS to quantitatively determine sucralose-6-acetate content in various sucralose-containing mediums.

Embodiments of the present disclosure relate to HPLC-based analytical methods that use MS to quantitatively determine sucralose-6-acetate content of a sucralose-containing medium, wherein the sucralose-containing medium is selected from the group consisting of a powder, a granular solid, a tablet, a crystalline solid, a liquid, a food, and a beverage.

Embodiments of the present disclosure relate to HPLC-based analytical methods that use MS/MS to quantitatively determine sucralose-6-acetate content of a sucralose-containing medium, wherein the sucralose-containing medium is selected from the group consisting of a powder, a granular solid, a tablet, a crystalline solid, a liquid, a food, and a beverage.

Embodiments of the present disclosure relate to UHPLC-based analytical methods that use MS to quantitatively determine sucralose-6-acetate content of a sucralose-containing medium, wherein the sucralose-containing medium is selected from the group consisting of a powder, a granular solid, a tablet, a crystalline solid, a liquid, a food, and a beverage.

Embodiments of the present disclosure relate to UHPLC-based analytical methods that use MS/MS to quantitatively determine sucralose-6-acetate content of a sucralose-containing medium, wherein the sucralose-containing medium is selected from the group consisting of a powder, a granular solid, a tablet, a crystalline solid, a liquid, a food, and a beverage.

Embodiments of the present disclosure relate to HPLC-based analytical methods that use MS to quantitatively determine sucralose-6-acetate content in a sucralose-containing medium, wherein the sucralose-containing medium contain one or both of dextrose and maltodextrin.

Embodiments of the present disclosure relate to HPLC-based analytical methods that use MS/MS to quantitatively determine sucralose-6-acetate content in a sucralose-containing medium, wherein the sucralose-containing medium contain one or both of dextrose and maltodextrin

Embodiments of the present disclosure relate to UHPLC-based analytical methods that use MS to quantitatively determine sucralose-6-acetate content in a sucralose-containing medium, wherein the sucralose-containing medium contain one or both of dextrose and maltodextrin

Embodiments of the present disclosure relate to UHPLC-based analytical methods that use MS/MS to quantitatively determine sucralose-6-acetate content in a sucralose-containing medium, wherein the sucralose-containing medium contain one or both of dextrose and maltodextrin

Embodiments of the present disclosure relate to HPLC-based analytical methods that use MS to quantitatively determine sucralose-6-acetate content in various sucralose-containing mediums that contain an amount of sucralose-6-acetate between about 1 ppb and about 10,000 ppm, between about 1 ppb and about 1,000 ppm, between about 1 ppb and about 100 ppm, between about 1 ppb and about 10 ppm, between about 1 ppb and about 1 ppm, between about 1 ppb and about 100 ppb, or between about 1 ppb and about 10 ppb.

Embodiments of the present disclosure relate to HPLC-based analytical methods that use MS/MS to quantitatively determine sucralose-6-acetate content in various sucralose-containing mediums that contain an amount of sucralose-6-acetate between about 1 ppb and about 10,000 ppm, between about 1 ppb and about 1,000 ppm, between about 1 ppb and about 100 ppm, between about 1 ppb and about 10 ppm, between about 1 ppb and about 1 ppm, between about 1 ppb and about 100 ppb, or between about 1 ppb and about 10 ppb.

Embodiments of the present disclosure relate to UHPLC-based analytical methods that use MS to quantitatively determine sucralose-6-acetate content in various sucralose-containing mediums that contain an amount of sucralose-6-acetate between about 1 ppb and about 10,000 ppm, between about 1 ppb and about 1,000 ppm, between about 1 ppb and about 100 ppm, between about 1 ppb and about 10 ppm, between about 1 ppb and about 1 ppm, between about 1 ppb and about 100 ppb, or between about 1 ppb and about 10 ppb.

Embodiments of the present disclosure relate to UHPLC-based analytical methods that use MS/MS to quantitatively determine sucralose-6-acetate content in various sucralose-containing mediums that contain an amount of sucralose-6-acetate between about 1 ppb and about 10,000 ppm, between about 1 ppb and about 1,000 ppm, between about 1 ppb and about 100 ppm, between about 1 ppb and about 10 ppm, between about 1 ppb and about 1 ppm, between about 1 ppb and about 100 ppb, or between about 1 ppb and about 10 ppb.

Embodiments of the present disclosure relate to HPLC-based analytical methods that use MS to quantitatively determine sucralose-6-acetate content in various sucralose-containing mediums that utilize a reverse phase column selected from the group consisting of C30 column, a C18 column, a C8 column, a C4 column, a polar-endcapped C18 column, an amide-embedded C18 column, a sulfonamide embedded C18 column, a phenyl column, a phenyl-hexyl column, a biphenyl column, and a pentafluorophenyl column.

Embodiments of the present disclosure relate to HPLC-based analytical methods that use MS/MS to quantitatively determine sucralose-6-acetate content in various sucralose-containing mediums that utilize a reverse phase column selected from the group consisting of C30 column, a C18 column, a C8 column, a C4 column, a polar-endcapped C18 column, an amide-embedded C18 column, a sulfonamide embedded C18 column, a phenyl column, a phenyl-hexyl column, a biphenyl column, and a pentafluorophenyl column.

Embodiments of the present disclosure relate to UHPLC-based analytical methods that use MS to quantitatively determine sucralose-6-acetate content in various sucralose-containing mediums that utilize a reverse phase column selected from the group consisting of C30 column, a C18 column, a C8 column, a C4 column, a polar-endcapped C18 column, an amide-embedded C18 column, a sulfonamide embedded C18 column, a phenyl column, a phenyl-hexyl column, a biphenyl column, and a pentafluorophenyl column.

Embodiments of the present disclosure relate to UHPLC-based analytical methods that use MS/MS to quantitatively determine sucralose-6-acetate content in various sucralose-containing mediums that utilize a reverse phase column selected from the group consisting of C30 column, a C18 column, a C8 column, a C4 column, a polar-endcapped C18 column, an amide-embedded C18 column, a sulfonamide embedded C18 column, a phenyl column, a phenyl-hexyl column, a biphenyl column, and a pentafluorophenyl column.

Embodiments of the present disclosure relate to HPLC-based analytical methods that use MS to quantitatively determine sucralose-6-acetate content in various sucralose-containing mediums that utilize a hydrophilic interaction column.

Embodiments of the present disclosure relate to HPLC-based analytical methods that use MS/MS to quantitatively determine sucralose-6-acetate content in various sucralose-containing mediums that utilize a hydrophilic interaction column.

Embodiments of the present disclosure relate to UHPLC-based analytical methods that use MS to quantitatively determine sucralose-6-acetate content in various sucralose-containing mediums that utilize a hydrophilic interaction column.

Embodiments of the present disclosure relate to UHPLC-based analytical methods that use MS/MS to quantitatively determine sucralose-6-acetate content in various sucralose-containing mediums that utilize a hydrophilic interaction column.

These and other features, aspects, and advantages of the present embodiments of the disclosure will become understood with reference to the following description, appended claims, and accompanying FIGURES.

### BRIEF DESCRIPTION OF THE DRAWINGS

FIG. 1 Sucralose-6-acetate content of commercial sucralose samples that were analyzed using a UHPLC-MS/MS analytical method, as described herein. In FIG. 1, the sucralose-6-acetate content is reported as ng of sucralose-6-acetate per g of product, or parts per billion (ppb), and was determined by selective ion monitoring (“SIM”), which is presented as black bars, and multiple reaction monitoring (“MRM”), which is presented as grey bars. For sucralose-6-acetate content determined by SIM, the sucralose-6-acetate content was determined by summing the areas of peaks that had a m/z that is characteristic of sucralose-6-acetate or adducts thereof, i.e., 437, 439, 473, 475, 477, 483, 485, 500, 502, 527, 529, 535, 537, 539, 589, 591, 593, 629, 631, 633, 747, 749, 854, 856, 858, 892, and 894. For sucralose-6-acetate content determined by MRM, sucralose-6-acetate content was determined by summing the areas of peaks that had a m/z transition that is characteristic of sucralose-6-acetate or adducts thereof, i.e., 473 to 437, 475 to 439, 483 to 437, and 485 to 439.

### DETAILED DESCRIPTION

Some embodiments provide an analytical method for quantitatively determining an amount of sucralose-6-acetate in a sucralose-containing material. As used herein, the term, “sucralose-containing material” refers to any material that contains the compound sucralose, regardless of the sucralose content of said material. The particular form of a sucralose-containing material is not particularly limited.

In some embodiments, a sucralose-containing material, as described herein, may define pure sucralose in a solid form, such as, for example, a powder, a granular solid, a crystalline solid, and the like.

In certain embodiments, sucralose-containing material, as described herein, may define pure sucralose in a liquid form, such as, for example, sucralose dissolved in water, a buffered liquid, or an aqueous solvent, and the like.

In some embodiments, a sucralose-containing material, as described herein, may define a solid material that contains sucralose, such as, for example, a solid form artificial sweetener, a solid form food additive, a solid food, a solid functional food, a solid dosage form nutritional product, a solid dosage form supplement, a solid dosage form nootropic, a solid dosage form nutraceutical, a solid dosage form medicine. In certain embodiments, solid dosage forms may include, but are not limited to a powder, a granular solid, a tablet, a capsule (soft, hard, gel, or plant-based), a pill, a caplet, a dissolvable oral strip, a hydrogel, a sachet, a gum, and the like.

In certain embodiments, a sucralose-containing material, as described herein, may define a liquid material that contains sucralose, such as, for example, a liquid form artificial sweetener, a liquid food additive, drinks/beverages, a liquid food, a liquid functional food, a liquid dosage form nutritional product, a liquid dosage form supplement, a liquid dosage form nootropic, a liquid dosage form nutraceutical, a liquid dosage form medicine. In certain embodiments, liquid dosage forms may include, but are not limited to a dispersion, a suspension, an aqueous solution, an oil-based solution, a beverage, a syrup, an elixir, an emulsion, and the like.

In some embodiments, solid and liquid form sucralose-containing materials, as described herein, may further comprise active ingredients, such as, for example, (i) active pharmaceutical ingredients; (ii) non-pharmaceutical active ingredients, such as, for example, caffeine, taurine, and the like; (iii) essential fatty acids, including, but not limited to linolenic acid, linoleic acid, and the like; (iv) essential amino acids, including, but not limited to tryptophan, lysine, methionine, phenylalanine, threonine, valine, leucine, isoleucine, arginine, and histidine, n-acetyl cysteine, and the like; (v) vitamins, including, but not limited to retinol (vitamin A), thiamine (vitamin B1), riboflavin (vitamin B2), niacin (vitamin B3), pantothenic acid (vitamin B5), pyridoxine, pyridoxamine, or pyridoxal (vitamin B6), biotin (vitamin B7) or pharmaceutically acceptable salts thereof, folic acid (vitamin B9) or pharmaceutically acceptable salts thereof, cobalamin (vitamin B12), choline, ascorbic acid (vitamin C) or pharmaceutically acceptable salts thereof, ergocalciferol (vitamin D2), calciferol (vitamin D3), 22-dihydroergocalciferol (vitamin D4), sitocalciferol (vitamin D5), tocopherol (vitamin E), phylloquinone (vitamin K1), menaquinone (vitamin K2), menadione (vitamin K3), and the like; (vi) dietary minerals, including, but not limited to chromium, bromine, cobalt, copper, fluorine, germanium, iodine, iron, magnesium, manganese, molybdenum, potassium, selenium, silicon, zinc, calcium, phosphorous, sodium, sulfur, vanadium, and the like; (vii) nitrates, including, but not limited to citrulline nitrate, creatine nitrate, beta-alanine nitrate, and the like; (viii) other dietary ingredients not specifically encompassed by any of the foregoing, including, but not limited to cranberry extract, turmeric, royal jelly, açaí berry, beet root, coral calcium, oyster shell, Gotu kola, Gingko biloba, lions mane mushroom, pomegranate, hibiscus flower, strawberry powder, dandelion root, celery powder, parsley powder, peppermint leaf, cinnamon bark powder, maca root, nicotinamide riboside, NAD+ precursors, Coenzyme Q10, omega-3-fatty acids, cabbage powder, nicotinamide mononucleotide, and the like; and (ix) any other compound, such as a vitamin, a mineral, an herb, a botanical, an amino acid, an enzyme, a probiotic, etc., that is not explicitly listed above, as well as any concentrates, metabolites, constituents, or extracts of any of the foregoing.

In certain embodiments, solid form and liquid form sucralose-containing materials, as described herein, may further comprise excipients, including, but not limited to (i) acidifying agents, such as, for example, acetic acid, glacial acetic acid, citric acid, fumaric acid, hydrochloric acid, diluted hydrochloric acid, malic acid, nitric acid, phosphoric acid, diluted phosphoric acid, sulfuric acid, tartaric acid, and the like; (ii) alkalizing agents, such as, for example, ammonia solution, ammonium carbonate, diethanolamine, diisopropanolamine, potassium hydroxide, sodium bicarbonate, sodium borate, sodium carbonate, sodium hydroxide, trolamine, and the like; (iii) antifoaming agents, such as, for example, dimethicone, simethicone, and the like; (iv) antimicrobial preservatives, such as, for example, benzalkonium chloride, benzalkonium chloride solution, benzethonium chloride, benzoic acid, benzyl alcohol, butylparaben, cetylpyridinium chloride, chlorobutanol, chlorocresol, cresol, dehydroacetic acid, ethylparaben, methylparaben, methylparaben sodium, phenol, phenylethyl alcohol, phenylmercuric acetate, phenylmercuric nitrate, potassium benzoate, potassium sorbate, propylparaben, propylparaben sodium, sodium benzoate, sodium dehydroacetate, sodium propionate, sorbic acid, thimerosal, thymol, and the like; (v) antioxidants, such as, for example, ascorbic acid, ascorbyl palmitate, butylated hydroxyanisole, butylated hydroxytoluene, hypophosphorous acid, monothioglycerol, propyl gallate, sodium formaldehyde sulfoxylate, sodium metabisulfite, sodium thiosulfate, sulfur dioxide, tocopherol, tocopherols excipient, and the like; (vi) buffering agents, such as, for example, acetic acid, ammonium carbonate, ammonium phosphate, boric acid, citric acid, lactic acid, phosphoric acid, potassium citrate, potassium metaphosphate, potassium phosphate monobasic, sodium acetate, sodium citrate, sodium lactate solution, dibasic sodium phosphate, monobasic sodium phosphate, and the like; (vii) chelating agents, such as, for example, edetate disodium, ethylenediaminetetraacetic acid and salts, edetic acid, and the like; (viii) coating agents, such as, for example, sodium carboxymethylcellulose, cellulose acetate, cellulose acetate phthalate, ethylcellulose, gelatin, pharmaceutical glaze, hydroxypropyl cellulose, hydroxypropyl methylcellulose, hydroxypropyl methylcellulose phthalate, methacrylic acid copolymer, methylcellulose, polyvinyl acetate phthalate, shellac, sucrose, titanium dioxide, carnauba wax, microcrystalline wax, zein, and the like; (ix) colorants, such as, for example, caramel, red, yellow, black or blends, ferric oxide, and the like; (x) complexing agents, such as, for example, ethylenediaminetetraacetic acid and salts (EDTA), edetic acid, gentisic acid ethanolamide, oxyquinoline sulfate, and the like; (xi) desiccants, such as, for example, calcium chloride, calcium sulfate, silicon dioxide, and the like; (xii) emulsifying and/or solubilizing agents, such as, for example, acacia, cholesterol, diethanolamine (adjunct), glyceryl monostearate, lanolin alcohols, mono- and di-glycerides, monoethanolamine (adjunct), lecithin, oleic acid (adjunct), oleyl alcohol (stabilizer), poloxamer, polyoxyethylene 50 stearate, polyoxyl 35 castor oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, diacetate, monostearate, sodium lauryl sulfate, sodium stearate, sorbitan monolaurate, sorbitan monooleate, sorbitan monopalmitate, sorbitan monostearate, stearic acid, trolamine, emulsifying wax, and the like; (xiii) filtering aids, such as, for example, powdered cellulose, purified siliceous earth, and the like; (xiv) flavors and perfumes, such as, for example, anethole, benzaldehyde, ethyl vanillin, menthol, methyl salicylate, monosodium glutamate, orange flower oil, peppermint, peppermint oil, peppermint spirit, rose oil, stronger rose water, thymol, tolu balsam tincture, vanilla, vanilla tincture, vanillin, and the like; (xv) humectants, such as, for example, glycerol, hexylene glycol, sorbitol, and the like; (xvi) plasticizers, such as, for example, castor oil, diacetylated monoglycerides, diethyl phthalate, glycerol, mono- and di-acetylated monoglycerides, propylene glycol, triacetin, triethyl citrate, and the like; (xvii) polymers, such as, for example, cellulose acetate, alkyl celluloses, hydroxyalkyl, acrylic polymers and copolymers, and the like; (xviii) solvents, such as, for example, acetone, alcohol, diluted alcohol, amylene hydrate, benzyl benzoate, butyl alcohol, carbon tetrachloride, chloroform, corn oil, cottonseed oil, dimethyl sulfoxide, ethyl acetate, glycerol, hexylene glycol, isopropyl alcohol, methyl alcohol, methylene chloride, methyl isobutyl ketone, mineral oil, peanut oil, propylene carbonate, sesame oil, water for injection, sterile water for injection, sterile water for irrigation, purified water, and the like; (xix) sorbents, such as, for example, powdered cellulose, charcoal, purified siliceous earth, and the like; (xx) carbon dioxide sorbents, such as, for example, barium hydroxide lime, soda lime, and the like; (xxi) stiffening agents, such as, for example, hydrogenated castor oil, cetostearyl alcohol, cetyl alcohol, cetyl esters wax, hard fat, paraffin, polyethylene excipient, stearyl alcohol, emulsifying wax, white wax, yellow wax, and the like; (xxii) suspending and/or thickening agents, such as, for example, acacia, agar, alginic acid, aluminum monostearate, bentonite, purified bentonite, magma bentonite, carbomer, carboxymethylcellulose calcium, carboxymethylcellulose sodium, carboxymethylcellulose sodium 12, carrageenan, microcrystalline and carboxymethylcellulose sodium cellulose, dextrin, gelatin (Bloom strength 50-100), guar gum, hydroxyethyl cellulose, hydroxypropyl cellulose, hydroxypropyl methylcellulose, magnesium aluminum silicate, methylcellulose, pectin, polyethylene oxide, polyvinyl alcohol, povidone, alginate, silicon dioxide, colloidal silicon dioxide, sodium alginate, tragacanth, xanthan gum, and the like; (xxiii) sweetening agents, such as, for example, aspartame, dextrates, dextrose, excipient dextrose, maltodextrin, fructose, mannitol, saccharin, calcium saccharin, sodium saccharin, sorbitol, solution sorbitol, sucrose, compressible sugar, confectioner's sugar, syrup, and the like; (xxiv) surfactants, such as, for example, simethicone, and the like; (xxv) tablet binders, such as, for example, acacia, alginic acid, sodium carboxymethylcellulose, microcrystalline cellulose, dextrin, ethylcellulose, gelatin, liquid glucose, guar gum, hydroxypropyl methylcellulose, methylcellulose, polyethylene oxide, povidone, pregelatinized starch, syrup, and the like; (xxvi) tablet and/or capsule diluents, such as, for example, calcium carbonate, dibasic calcium phosphate, tribasic calcium phosphate, calcium sulfate, microcrystalline cellulose, powdered cellulose, dextrates, dextrin, dextrose excipient, fructose, kaolin, lactose, mannitol, sorbitol, starch, pregelatinized starch, sucrose, compressible sugar, confectioner's sugar, and the like; (xxvii) tablet disintegrants, such as, for example, alginic acid, microcrystalline cellulose, croscarmellose sodium, crospovidone, polacrilin potassium, sodium starch glycolate, starch, pregelatinized starch, and the like; (xxviii) tablet and/or capsule lubricants, such as, for example, calcium stearate, glyceryl behenate, magnesium stearate, light mineral oil, sodium stearyl fumarate, stearic acid, purified stearic acid, talc, hydrogenated vegetable oil, zinc stearate, and the like; (xxix) tonicity agent, such as, for example, dextrose, glycerol, mannitol, potassium chloride, sodium chloride, and the like; (xxx) vehicle: flavored and/or sweetened, such as, for example, aromatic elixir, compound benzaldehyde elixir, iso-alcoholic elixir, peppermint water, sorbitol solution, syrup, tolu balsam syrup, and the like; (xxxi) vehicle: oleaginous, such as, for example, almond oil, corn oil, cottonseed oil, ethyl oleate, isopropyl myristate, isopropyl palmitate, mineral oil, light mineral oil, myristyl alcohol, octyl dodecanol, olive oil, peanut oil, persic oil, sesame oil, soybean oil, squalane, and the like; (xxxii) vehicle: solid carrier, such as, for example, sugar spheres, and the like; (xxxiii) vehicle: sterile, such as, for example, bacteriostatic water for injection, bacteriostatic sodium chloride injection, and the like; (xxxiv) water repelling agents, such as, for example, cyclomethicone, dimethicone, simethicone, and the like; and/or (xxxv) solubilizing agent, such as, for example, benzalkonium chloride, benzethonium chloride, cetylpyridinium chloride, docusate sodium, nonoxynol 9, nonoxynol 10, octoxynol 9, poloxamer, polyoxyl 35 castor oil, polyoxyl 40, hydrogenated castor oil, polyoxyl 50 stearate, polyoxyl 10 oleyl ether, polyoxyl 20 cetostearyl ether, polyoxyl 40 stearate, polysorbate 20, polysorbate 40, polysorbate 60, polysorbate 80, sodium lauryl sulfate, sorbitan monolaurate, sorbitan monooleate, sorbitan monopalmitate, sorbitan monostearate, tyloxapol, and the like. This list is not meant to be exclusive, but instead merely representative of the classes of excipients that may be used in the formulations described herein. This list is also not meant to do exclude a compound from satisfying multiple excipient purposes—for example, dextrose and maltodextrin may be provided in some sucralose-containing materials as sweeteners, and as diluents in others; regardless of the particular specified excipient use, the methods described herein would nonetheless apply to either.

In some embodiments, an analytical method for quantitatively determining an amount of sucralose-6-acetate in a sucralose-containing material, as described herein, may provide a liquid chromatography separation unit configured to perform liquid chromatography. The components that may define a liquid chromatography separation unit, as described herein, are not particularly limited-exemplary components of a liquid chromatography separation unit, as described herein, may include, but are not limited to (i) one or more controllers; (ii) one or more processors; (iii) a computer readable medium; (iv) tubing configured to transport liquid therethrough; (v) a chromatographic column configured to connect to tubing at the entrance and the exit of the chromatographic column; (vi) a mobile phase comprising one or more liquid solvents; (vii) one or more pumps configured to pump the mobile phase through tubing and the chromatographic column; and (viii) a means for injecting one or more liquid samples sequentially into the liquid chromatography unit at a location upstream of the entrance of the chromatographic column.

In certain embodiments, a liquid chromatography separation unit, as described herein, can be configured as an HPLC Unit. As used herein, the term “HPLC Unit” refers to a liquid chromatography separation unit, as described herein, that is configured to perform high-performance liquid chromatography (HPLC).

In some embodiments, an HPLC Unit, as described herein may further comprise a temperature-controlled oven. In certain embodiments, a temperature-controlled oven, as described herein, can be configured to store and maintain a temperature of the chromatographic column, as described herein, while the HPLC Unit is operating. In some embodiments, a temperature-controlled oven, as described herein, can maintain the temperature of the chromatographic column, as described herein, at a temperature of about 20° C. to about 90° C., or any fraction or integer therebetween.

In certain embodiments, an HPLC Unit, as described herein, may comprise an autosampler configured as the means for injecting one or more liquid samples into the HPLC Unit at a location upstream of the entrance of the chromatographic column, as described herein. In some embodiments, an autosampler, as described herein, may be configured to store and maintain the temperature of one or more samples prior to injection into the HPLC Unit. In certain embodiments, an autosampler, as described herein, can maintain the temperature of one or more samples at a temperature of about 4° C. to about 20° C., or any fraction or integer therebetween.

In certain embodiments, an HPLC Unit, as described herein, may be configured to be operated at a flowrate of about 0.2 mL/min to about 2.0 mL/min, or any fraction or integer therebetween.

In some embodiments, an HPLC Unit, as described herein, may be configured to accept an injection volume of about 1.0 μL to about 500 μL, or any fraction or integer therebetween.

In certain embodiments, an HPLC Unit, as described herein, may be configured to be operated with a back pressure of about 50 bar to about 600 bar, or any fraction or integer therebetween.

In some embodiments, the chromatographic column of an HPLC Unit, as described herein, may be a reversed-phase chromatographic column (“RP-HPLC Column”).

In certain embodiments, a RP-HPLC Column, as described herein, may be a C30 column, a C18 column, a C8 column, a C4 column, a polar-endcapped C18 column, an amide-embedded C18 column, a sulfonamide embedded C18 column, a phenyl column, a phenyl-hexyl column, a biphenyl column, or a pentafluorophenyl column. In some embodiments, a RP-HPLC Column, as described herein, may preferably be a C18 column.

In some embodiments, a RP-HPLC Column, as described herein, may comprise a particle size of about 2.7 μm to about 5 μm.

In certain embodiments, a RP-HPLC Column, as described herein, may comprise an inner diameter of about 2.0 mm to about 4.6 mm.

In some embodiments, a RP-HPLC Column, as described herein, may comprise a column length of about 50 mm to about 300 mm.

In certain embodiments, the chromatographic column of an HPLC Unit, as described herein, may be a hydrophilic interaction chromatographic column (“HILIC-HPLC Column”).

In some embodiments, a HILIC-HPLC Column, as described herein, may comprise a particle size of about 2.7 μm to about 5 μm.

In certain embodiments, a HILIC-HPLC Column, as described herein, may comprise an inner diameter of about 2.0 mm to about 4.6 mm.

In some embodiments, a HILIC-HPLC Column, as described herein, may comprise a column length of about 50 mm to about 300 mm.

In certain embodiments, a liquid chromatography separation unit, as described herein, can be configured as a UHPLC Unit. As used herein, the term “UHPLC Unit” refers to a liquid chromatography separation unit, as described herein, that is configured to perform ultra-high-performance liquid chromatography (UHPLC).

In some embodiments, a UHPLC Unit, as described herein may further comprise a temperature-controlled oven. In certain embodiments, a temperature-controlled oven, as described herein, can be configured to store and maintain a temperature of the chromatographic column, as described herein, while the UHPLC Unit is operating. In some embodiments, a temperature-controlled oven, as described herein, can maintain the temperature of the chromatographic column, as described herein, at a temperature of about 20° C. to about 90° C., or any fraction or integer therebetween.

In certain embodiments, a UHPLC Unit, as described herein, may comprise an autosampler configured as the means for injecting one or more liquid samples into the UHPLC Unit at a location upstream of the entrance of the chromatographic column, as described herein. In some embodiments, an autosampler, as described herein, may be configured to store and maintain the temperature of one or more samples prior to injection into the UHPLC Unit. In certain embodiments, an autosampler, as described herein, can maintain the temperature of one or more samples at a temperature of about 4° C. to about 20° C., or any fraction or integer therebetween.

In certain embodiments, a UHPLC Unit, as described herein, may be configured to be operated at a flowrate of about 0.2 mL/min to about 0.7 mL/min, or any fraction or integer therebetween.

In some embodiments, a UHPLC Unit, as described herein, may be configured to accept an injection volume of about 0.2 μL to about 100 μL, or any fraction or integer therebetween.

In certain embodiments, a UHPLC Unit, as described herein, may be configured to be operated with a back pressure of up to about 1500 bar.

In some embodiments, the chromatographic column of a UHPLC Unit, as described herein, may be a reversed-phase chromatographic column (“RP-UHPLC Column”).

In certain embodiments, a RP-UHPLC Column, as described herein, may be a C30 column, a C18 column, a C8 column, a C4 column, a polar-endcapped C18 column, an amide-embedded C18 column, a sulfonamide embedded C18 column, a phenyl column, a phenyl-hexyl column, a biphenyl column, or a pentafluorophenyl column. In some embodiments, a RP-UHPLC Column, as described herein, may preferably be a C18 column.

In some embodiments, a RP-UHPLC Column, as described herein, may comprise a particle size of less than about 2.0 μm. In certain embodiments, a RP-UHPLC Column, as described herein, may preferably comprise a particle size of about 1.5 μm to about 2.0 μm.

In some embodiments, a RP-UHPLC Column, as described herein, may comprise an inner diameter of about 1.0 mm to about 4.6 mm. In certain embodiments, a RP-UHPLC Column, as described herein, may preferably comprise an inner diameter of about 1.0 mm to about 2.1 mm.

In some embodiments, a RP-UHPLC Column, as described herein, may comprise a column length of about 30 mm to about 150 mm. In certain embodiments, a RP-UHPLC Column, as described herein, may preferably comprise a column length of about 50 mm to about 100 mm.

In certain embodiments, the chromatographic column of a UHPLC Unit, as described herein, may be a hydrophilic interaction chromatographic column (“HILIC-UHPLC Column”).

In some embodiments, a HILIC-UHPLC Column, as described herein, may comprise a particle size of less than about 2.0 μm. In certain embodiments, a HILIC-UHPLC Column, as described herein, may preferably comprise a particle size of about 1.5 μm to about 2.0 μm.

In some embodiments, a HILIC-UHPLC Column, as described herein, may comprise an inner diameter of about 1.0 mm to about 4.6 mm. In certain embodiments, a HILIC-UHPLC Column, as described herein, may preferably comprise an inner diameter of about 1.0 mm to about 2.1 mm.

In some embodiments, a HILIC-UHPLC Column, as described herein, may comprise a column length of about 30 mm to about 150 mm. In certain embodiments, a HILIC-UHPLC Column, as described herein, may preferably comprise a column length of about 50 mm to about 100 mm.

In certain embodiments, an HPLC Unit and/or a UHPLC Unit, as described herein, may further comprise a detection means provided downstream of the chromatographic column, as described herein, configured to receive an eluent from the chromatographic column. In some embodiments, a detection means, as described herein, may be configured measure a property of the eluent, as described herein, and produce measurement data. In certain embodiments, a detection means, as described herein, may be configured to transmit measurement data, as described herein, to the HPLC Unit and/or the UHPLC Unit.

In some embodiments, a detection means, as described herein, may define an ultraviolet-visible light (UV-vis) detector configured to measure an absorbance of the eluent at one or more wavelengths in the range of 190-800 nm and produce absorbance measurement data.

In certain embodiments, a detection means, as described herein, may define a fluorescence detector configured to measure a fluorescence of the eluent at one or more emission and excitation wavelengths in the range of 200-630 nm and produce fluorescence measurement data.

In some embodiments, a detection means, as described herein, may define a refractive index detector configured to measure a refractive index of the eluent and produce refractive index measurement data.

In certain embodiments, a detection means, as described herein, may define an electrochemical detector configured to measure a current of the eluent produced by oxidation or reduction at a surface of an electrode contained within the electrochemical detector and produce current measurement data.

In some embodiments, a detection means, as described herein, may define a charged aerosol detector configured to measure an aggregate charge of the eluent produced by nebulization the eluent and charging the nebulized eluent with an ionized gas and produce aggregate charge measurement data.

In some embodiments, a detection means, as described herein, may define any combination of the foregoing.

In certain embodiments, an HPLC Unit and/or a UHPLC Unit, as described herein, may be configured to generate a chromatogram from measurement data received from a detection means, as described herein. As used herein, the term “chromatogram” refers to a visual representation, e.g., a graph, of the measurement data as a function of time, wherein temporal changes in measurement data generate one or more peaks, each comprising a peak area, and corresponding to one or more compounds in the eluent.

In some embodiments, an HPLC Unit and/or a UHPLC Unit, as described herein, may be configured to determine peak area of all peaks present on a chromatogram, as described herein. In certain embodiments, peak areas, as described herein, may be used to quantitatively determine an amount of one or more compounds that are not sucralose-6-acetate in a sucralose-containing material, as described herein; such compounds include, but are not limited to active ingredients, excipients, or additives not explicitly listed present in a sucralose-containing material, as described herein. In some embodiments, a detection means, as described herein, may preferably be used to quantitatively determine an amount of a compound that is not sucralose-6-acetate present in a sucralose-containing material, as described herein, including, but not limited to sucralose, dextrose, maltodextrin, dextrin, as well as any other excipient or additive that would commonly be found in artificial sweeteners, food additives, foods, drinks/beverages, functional foods, nutritional products, supplements, nootropics, nutraceuticals, and/or medicines. In certain embodiments, a detection means, as described herein, may more preferably be used to quantitatively determine an amount of a compound that is not sucralose-6-acetate present in a sucralose-containing material, as described herein, including, but not limited to sucralose, dextrose, maltodextrin, dextrin, as well as any other excipient or additive that would commonly be found in artificial sweeteners, food additives, foods, drinks/beverages, and/or functional foods.

The particular methods for quantitatively determining an amount of a compound that is not sucralose-6-acetate present in a sucralose-containing material, as described herein, are not particularly limited, and may be analogous to other quantitative methods disclosed herein. One such, non-limiting, method for quantitatively determining an amount of a compound that is not sucralose-6-acetate present in a sucralose-containing material, as described herein, may be, for example: (i) preparing calibration curve samples for a compound of interest, e.g., sucralose, dextrose, and/or maltodextrin, and one or more sucralose-containing material samples containing compound of interest; (ii) determining a peak area for the compound of interest in each sample of the calibration curve samples; (iii) generating plotted data by plotting peak area of each calibration curve sample against its concentration; (iv) fitting the plotted data with a linear best-fit line; (v) determining a peak area of the compound of interest in each of the one or more sucralose-containing material samples containing compound of interest; (vi) calculating a concentration of the compound of interest in each of the one or more sucralose-containing material samples containing compound of interest from the linear best-fit line; and (vii) optionally, converting the concentration of the compound of interest in each of the one or more sucralose-containing material samples containing compound of interest to a mass or mass percent.

In certain embodiments, an HPLC Unit and/or a UHPLC Unit, as described herein, as described herein, may be configured to operate in compliance with standards set by one or more regulatory agencies. In certain embodiments, a regulatory agency, as described herein, may refer to the European Food Safety Authority (EFSA), the Environmental Protection Agency (EPA), the Food and Drug Administration (FDA), any combination of the foregoing, and the like. In some embodiments, an HPLC Unit and/or a UHPLC Unit, as described herein, may be configured to operate in compliance with the Good Laboratory Practice for Nonclinical Laboratory Studies, as that term is used in 21 C.F.R. § 58 et seq. In certain embodiments, an HPLC Unit and/or a UHPLC Unit, as described herein, may be configured to operate in compliance with the Quality Guidelines, as that term is used by the International Conference on Harmonization (ICH).

In some embodiments, an HPLC Unit and/or a UHPLC Unit, as described herein, as described herein, may be configured to implement a liquid chromatography method. In certain embodiments, a liquid chromatography method, as described herein, may specify (i) a run time; (ii) a composition of the mobile phase, as described herein, at one or more time points within the run time; and (iii) a flowrate of the mobile phase, as described herein. In some embodiments, a liquid chromatography method, as described herein, may specify that a composition of the mobile phase that is constant at each of the one or more time points defining the run time (“Isocratic Method”). In certain embodiments, a liquid chromatography method, as described herein, may specify that a composition of the mobile phase is different at two or more time points defining the run time (“Gradient Method”).

In some embodiments, an Isocratic Method, as described herein, may specify a run time that may be at least 5 minutes, at least 10 minutes, at least 15 minutes, at least 20 minutes, at least 25 minutes, at least 30 minutes, at least 35 minutes, at least 40 minutes, at least 45 minutes, at least 50 minutes, at least 55 minutes, at least 60 minutes, at least 65 minutes, at least 70 minutes, at least 75 minutes, at least 80 minutes, at least 85 minutes, at least 90 minutes, at least 95 minutes, at least 100 minutes, or any fraction or integer in between any two of the preceding amounts.

In certain embodiments, an Isocratic Method, as described herein, may specify a composition of a mobile phase, as described herein, defining a mixture of an aqueous solvent and an organic solvent. As used herein, the term, “aqueous solvent,” may refer to a water-based solvent, such as, for example, water, an aqueous buffer, and the like. Whether “aqueous solvent,” refers to water, an aqueous buffer, or an alternative solvent would be immediately envisaged by the skilled artisan when viewing the term in the context of the particular solvent disclosed and whether an HPLC Unit and/or a UHPLC Unit, as described herein, is referenced therewith. As used herein, the term, “organic solvent,” may refer an organic solvent, such as, for example, acetonitrile, methanol, ethanol, isopropanol, n-propanol, dimethylsulfoxide, dimethylformamide, tetrahydrofuran, acetone, ethyl acetate, hexane, heptane, and the like. Whether “organic solvent,” refers to any of the foregoing organic solvents or an alternative solvent would be immediately envisaged by the skilled artisan when viewing the term in the context of the particular solvent disclosed and whether an HPLC Unit and/or a UHPLC Unit, as described herein, is referenced therewith.

In some embodiments, an Isocratic Method, as described herein, may specify a composition of a mobile phase, as described herein, defining a mixture of an aqueous solvent and an organic solvent and further comprising one or more additives.

In certain embodiments, an additive, as described herein, may define an acid additive, such as, for example, formic acid, acetic acid, difluoroacetic acid, trifluoroacetic acid, pentafluoropropionic acid, heptafluorobutyric acid, methanesulphonic acid, and the like. In certain embodiments, a composition of a mobile phase, as described herein, may comprise an acid additive, as described herein, by volume, of up to about 0.01%, up to about 0.02%, up to about 0.03%, up to about 0.04%, up to about 0.05%, up to about 0.06%, up to about 0.07%, up to about 0.08%, up to about 0.09%, up to about 0.10%, up to about 0.20%, up to about 0.30%, up to about 0.40%, up to about 0.50%, up to about 0.60%, up to about 0.70%, up to about 0.80%, up to about 0.90%, up to about 1.0%, or any fraction or integer in between any two of the preceding amounts.

In some embodiments, an additive, as described herein, may define a non-acid additive, such as, for example, sodium formate, 2,2,2-trifluoroethanol, 1,1,1,3,3,3-hexafluoro-2-propanol, ammonium hydroxide, ammonium acetate, ammonium bicarbonate ammonium acetate/acetic acid buffer, ammonium formate/formic acid buffer, ammonia/ammonium hydroxide buffer, and the like. In some embodiments, a composition of a mobile phase, as described herein, may comprise a non-acid additive, as described herein, by volume, of up to about 0.01%, up to about 0.02%, up to about 0.03%, up to about 0.04%, up to about 0.05%, up to about 0.06%, up to about 0.07%, up to about 0.08%, up to about 0.09%, up to about 0.10%, up to about 0.20%, up to about 0.30%, up to about 0.40%, up to about 0.50%, up to about 0.60%, up to about 0.70%, up to about 0.80%, up to about 0.90%, up to about 1.0%, or any fraction or integer in between any two of the preceding amounts.

In certain embodiments, an Isocratic Method, as described herein, may specify a composition of a mobile phase, as described herein, that may comprise, by volume, at least 5% aqueous solvent and at most 95% organic solvent, as described herein, at least 10% aqueous solvent and at most 90% organic solvent, as described herein, at least 15% aqueous solvent and at most 85% organic solvent, as described herein, at least 20% aqueous solvent and at most 80% organic solvent, as described herein, at least 25% aqueous solvent and at most 75% organic solvent, as described herein, at least 30% aqueous solvent and at most 70% organic solvent, as described herein, at least 35% aqueous solvent and at most 65% organic solvent, as described herein, at least 40% aqueous solvent and at most 60% organic solvent, as described herein, at least 65% aqueous solvent and at most 35% organic solvent, as described herein, at least 40% aqueous solvent and at most 60% organic solvent, as described herein, at least 45% aqueous solvent and at most 55% organic solvent, as described herein, at least 60% aqueous solvent and at most 40% organic solvent, as described herein, at least 65% aqueous solvent and at most 35% organic solvent, as described herein, at least 70% aqueous solvent and at most 30% organic solvent, as described herein, at least 75% aqueous solvent and at most 25% organic solvent, as described herein, at least 80% aqueous solvent and at most 20% organic solvent, as described herein, at least 85% aqueous solvent and at most 15% organic solvent, as described herein, at least 90% aqueous solvent and at most 10% organic solvent, as described herein, at least 91% aqueous solvent and at most 9% organic solvent, as described herein, at least 92% aqueous solvent and at most 8% organic solvent, as described herein, at least 93% aqueous solvent and at most 7% organic solvent, as described herein, at least 94% aqueous solvent and at most 6% organic solvent, as described herein, at least 95% aqueous solvent and at most 5% organic solvent, as described herein, at least 96% aqueous solvent and at most 4% organic solvent, as described herein, at least 97% aqueous solvent and at most 3% organic solvent, as described herein, at least 98% aqueous solvent and at most 2% organic solvent, as described herein, at least 99% aqueous solvent and at most 1% organic solvent, as described herein, or any fraction or integer in between any two of the preceding amounts.

In some certain embodiments, an Isocratic Method, as described herein, may specify a composition of a mobile phase, as described herein, that may comprise, by volume, about 5% water, about 95% acetonitrile, methanol, or a mixture thereof, and about 0.1% formic acid, acetic acid, or a mixture thereof, about 10% water, about 90% acetonitrile, methanol, or a mixture thereof, and about 0.1% formic acid, acetic acid, or a mixture thereof, about 15% water, about 85% acetonitrile, methanol, or a mixture thereof, and about 0.1% formic acid, acetic acid, or a mixture thereof, about 20% water, about 80% acetonitrile, methanol, or a mixture thereof, and about 0.1% formic acid, acetic acid, or a mixture thereof, about 25% water, about 75% acetonitrile, methanol, or a mixture thereof, and about 0.1% formic acid, acetic acid, or a mixture thereof, about 30% water, about 70% acetonitrile, methanol, or a mixture thereof, and about 0.1% formic acid, acetic acid, or a mixture thereof, about 35% water, about 65% acetonitrile, methanol, or a mixture thereof, and about 0.1% formic acid, acetic acid, or a mixture thereof, about 40% water, about 60% acetonitrile, methanol, or a mixture thereof, and about 0.1% formic acid, acetic acid, or a mixture thereof, about 45% water, about 55% acetonitrile, methanol, or a mixture thereof, and about 0.1% formic acid, acetic acid, or a mixture thereof, about 50% water, about 50% acetonitrile, methanol, or a mixture thereof, and about 0.1% formic acid, acetic acid, or a mixture thereof, about 55% water, about 45% acetonitrile, methanol, or a mixture thereof, and about 0.1% formic acid, acetic acid, or a mixture thereof, about 60% water, about 40% acetonitrile, methanol, or a mixture thereof, and about 0.1% formic acid, acetic acid, or a mixture thereof, about 65% water, about 35% acetonitrile, methanol, or a mixture thereof, and about 0.1% formic acid, acetic acid, or a mixture thereof, about 70% water, about 30% acetonitrile, methanol, or a mixture thereof, and about 0.1% formic acid, acetic acid, or a mixture thereof, about 75% water, about 25% acetonitrile, methanol, or a mixture thereof, and about 0.1% formic acid, acetic acid, or a mixture thereof, about 80% water, about 20% acetonitrile, methanol, or a mixture thereof, and about 0.1% formic acid, acetic acid, or a mixture thereof, about 85% water, about 15% acetonitrile, methanol, or a mixture thereof, and about 0.1% formic acid, acetic acid, or a mixture thereof, about 90% water, about 10% acetonitrile, methanol, or a mixture thereof, and about 0.1% formic acid, acetic acid, or a mixture thereof, about 91% water, about 9% acetonitrile, methanol, or a mixture thereof, and about 0.1% formic acid, acetic acid, or a mixture thereof, about 92% water, about 8% acetonitrile, methanol, or a mixture thereof, and about 0.1% formic acid, acetic acid, or a mixture thereof, about 93% water, about 7% acetonitrile, methanol, or a mixture thereof, and about 0.1% formic acid, acetic acid, or a mixture thereof, about 94% water, about 6% acetonitrile, methanol, or a mixture thereof, and about 0.1% formic acid, acetic acid, or a mixture thereof, about 95% water, about 5% acetonitrile, methanol, or a mixture thereof, and about 0.1% formic acid, acetic acid, or a mixture thereof, about 96% water, about 4% acetonitrile, methanol, or a mixture thereof, and about 0.1% formic acid, acetic acid, or a mixture thereof, about 97% water, about 3% acetonitrile, methanol, or a mixture thereof, and about 0.1% formic acid, acetic acid, or a mixture thereof, about 98% water, about 2% acetonitrile, methanol, or a mixture thereof, and about 0.1% formic acid, acetic acid, or a mixture thereof, about 99% water, about 1% acetonitrile, methanol, or a mixture thereof, and about 0.1% formic acid, acetic acid, or a mixture thereof, or any fraction or integer in between any two of the preceding amounts.

In some embodiments, an Isocratic Method, as described herein, may further include a washing step that occurs after the run time. In certain embodiments, a washing step, as described herein, may specify a washing step run time and a washing step composition. In some embodiments, a washing step run time, as described herein, that may be at least 1 minute, at least 5 minutes, at least 10 minutes, at least 15 minutes, at least 20 minutes, at least 25 minutes, at least 30 minutes, at least 35 minutes, at least 40 minutes, at least 45 minutes, at least 50 minutes, at least 55 minutes, at least 60 minutes, at least 65 minutes, at least 70 minutes, at least 75 minutes, at least 80 minutes, at least 85 minutes, at least 90 minutes, at least 95 minutes, at least 100 minutes, or any fraction or integer in between any two of the preceding amounts. In certain embodiments, a washing step composition, as described herein, may comprise, by volume, at least 90% organic solvent and at most 10% aqueous solvent, as described herein, at least 91% organic solvent and at most 9% aqueous solvent, as described herein, at least 92% organic solvent and at most 8% aqueous solvent, as described herein, at least 93% organic solvent and at most 7% aqueous solvent, as described herein, at least 94% organic solvent and at most 6% aqueous solvent, as described herein, at least 95% organic solvent and at most 5% aqueous solvent, as described herein, at least 96% organic solvent and at most 4% aqueous solvent, as described herein, at least 97% organic solvent and at most 3% aqueous solvent, as described herein, at least 98% organic solvent and at most 2% aqueous solvent, as described herein, at least 99% organic solvent and at most 1% aqueous solvent, as described herein, entirely organic solvent, as described herein, or any fraction or integer in between any two of the preceding amounts.

In some embodiments, an Isocratic Method, as described herein, may provide a separation factor between sucralose-6-acetate and at least one other compound in a sucralose-containing material, as described herein, including, but not limited to sucralose, dextrose, maltodextrin, dextrin, as well as any other excipient or additive that would commonly be found in artificial sweeteners, food additives, foods, drinks/beverages, functional foods, nutritional products, supplements, nootropics, nutraceuticals, and/or medicines. In certain embodiments, an Isocratic Method, as described herein, may preferably provide a separation factor between sucralose-6-acetate and another compound in a sucralose-containing material, as described herein, including, but not limited to sucralose, dextrose, maltodextrin, dextrin, as well as any other excipient or additive that would commonly be found in artificial sweeteners, food additives, foods, drinks/beverages, and/or functional foods. As used herein, the term, “separation factor” refers to the difference in retention time between sucralose-6-acetate and at least one other compound in a sucralose-containing material, as described herein, when an Isocratic Method, as described herein, is implemented on an HPLC Unit and/or a UHPLC Unit, as described herein, for a sample comprising a sucralose-containing material, as described herein. In certain embodiments, an Isocratic Method, as described herein, may provide a separation factor between sucralose-6-acetate and at least one other compound in a sucralose-containing material, as described herein, of at least 0.5 minutes, at least 0.6 minutes, at least 0.7 minutes, at least 0.8 minutes, at least 0.9 minutes, at least 1.0 minute, at least 1.1 minutes, at least 1.2 minutes, at least 1.3 minutes, at least 1.4 minutes, at least 1.5 minutes, at least 1.6 minutes, at least 1.7 minutes, 1.8 minutes, at least 1.9 minutes, at least 2.0 minutes, at least 2.1 minutes, at least 2.2 minutes, at least 2.3 minutes, at least 2.4 minutes, at least 2.5 minutes, at least 2.6 minutes, at least 2.7 minutes, at least 2.8 minutes, at least 2.9 minutes, at least 3.0 minutes, at least 3.1 minutes, at least 3.2 minutes, at least 3.3 minutes, at least 3.4 minutes, at least 3.5 minutes, at least 3.6 minutes, at least 3.7 minutes, at least 3.8 minutes, at least 3.9 minutes, at least 4.0 minutes, at least 4.1 minutes, at least 4.2 minutes, at least 4.3 minutes, at least 4.4 minutes, at least 4.5 minutes, at least 4.6 minutes, at least 4.7 minutes, at least 4.8 minutes, at least 4.9 minutes, at least 5.0 minutes, or any fraction or integer in between any two of the preceding amounts.

In certain embodiments, a Gradient Method, as described herein, may specify a run time that may be at least 5 minutes, at least 10 minutes, at least 15 minutes, at least 20 minutes, at least 25 minutes, at least 30 minutes, at least 35 minutes, at least 40 minutes, at least 45 minutes, at least 50 minutes, at least 55 minutes, at least 60 minutes, at least 65 minutes, at least 70 minutes, at least 75 minutes, at least 80 minutes, at least 85 minutes, at least 90 minutes, at least 95 minutes, at least 100 minutes, or any fraction or integer in between any two of the preceding amounts. In some embodiments, a Gradient Method, as described herein, may preferably specify a run time that may be at most 40 minutes, at most 35 minutes, at most 30 minutes, at most 25 minutes, at most 20 minutes, at most 19 minutes, at most 18 minutes, at most 17 minutes, at most 16 minutes, at most 15 minutes, at most 14 minutes, at most 13 minutes, at most 12 minutes, at most 11 minutes, at most 10 minutes, or any fraction or integer in between any two of the preceding amounts.

In some embodiments, a Gradient Method, as described herein, may specify a composition of a mobile phase, as described herein, defining a mixture of a first liquid solvent (“Solvent A”) and a second liquid solvent (“Solvent B”). In certain embodiments, Solvent A and/or Solvent B, as described herein, may comprise a mixture of an aqueous solvent and an organic solvent, as described herein.

In certain embodiments, Solvent A, as described herein, may comprise entirely aqueous solvent, as described herein, by volume. In some embodiments, Solvent A, as described herein, may comprise more aqueous solvent than organic solvent, by volume, such as, for example, at least 70% aqueous solvent and at most 30% organic solvent, as described herein, at least 75% aqueous solvent and at most 25% organic solvent, as described herein, at least 80% aqueous solvent and at most 20% organic solvent, as described herein, at least at least 85% aqueous solvent and at most 15% organic solvent, as described herein, at least 90% aqueous solvent and at most 10% organic solvent, as described herein, at least 91% aqueous solvent and at most 9% organic solvent, as described herein, at least 92% aqueous solvent and at most 8% organic solvent, as described herein, at least 93% aqueous solvent and at most 7% organic solvent, as described herein, at least 94% aqueous solvent and at most 6% organic solvent, as described herein, at least 95% aqueous solvent and at most 5% organic solvent, as described herein, at least 96% aqueous solvent and at most 4% organic solvent, as described herein, at least 97% aqueous solvent and at most 3% organic solvent, as described herein, at least 98% aqueous solvent and at most 2% organic solvent, as described herein, at least 99% aqueous solvent and at most 1% organic solvent, as described herein, or any fraction or integer in between any two of the preceding amounts.

In some embodiments, Solvent B, as described herein, may comprise entirely organic solvent, as described herein, by volume. In certain embodiments, Solvent B, as described herein, may comprise more organic solvent than aqueous solvent, by volume, such as, for example, at least 70% organic solvent and at most 30% aqueous solvent, as described herein, at least 75% organic solvent and at most 25% aqueous solvent, as described herein, at least 80% organic solvent and at most 20% aqueous solvent, as described herein, at least at least 85% organic solvent and at most 15% aqueous solvent, as described herein, at least 90% organic solvent and at most 10% aqueous solvent, as described herein, at least 91% organic solvent and at most 9% aqueous solvent, as described herein, at least 92% organic solvent and at most 8% aqueous solvent, as described herein, at least 93% organic solvent and at most 7% aqueous solvent, as described herein, at least 94% organic solvent and at most 6% aqueous solvent, as described herein, at least 95% organic solvent and at most 5% aqueous solvent, as described herein, at least 96% organic solvent and at most 4% aqueous solvent, as described herein, at least 97% organic solvent and at most 3% aqueous solvent, as described herein, at least 98% organic solvent and at most 2% aqueous solvent, as described herein, at least 99% organic solvent and at most 1% aqueous solvent, as described herein, or any fraction or integer in between any two of the preceding amounts.

In certain embodiments, Solvent A and/or Solvent B, as described herein, may further comprise one or more additives, as described herein. In some embodiments, Solvent A and/or Solvent B, as described herein, may further comprise an additive, as described herein, by volume, of up to about 0.01%, up to about 0.02%, up to about 0.03%, up to about 0.04%, up to about 0.05%, up to about 0.06%, up to about 0.07%, up to about 0.08%, up to about 0.09%, up to about 0.10%, up to about 0.20%, up to about 0.30%, up to about 0.40%, up to about 0.50%, up to about 0.60%, up to about 0.70%, up to about 0.80%, up to about 0.90%, up to about 1.0%, or any fraction or integer in between any two of the preceding amounts.

In some embodiments, Solvent A, as described herein, may comprise about 90% water, about 10% acetonitrile, methanol, or a mixture thereof, and about 0.1% formic acid, acetic acid, or a mixture thereof. In some embodiments, Solvent A, as described herein, may comprise about 91% water, about 9% acetonitrile, methanol, or a mixture thereof, and about 0.1% formic acid, acetic acid, or a mixture thereof. In certain embodiments, Solvent A, as described herein, may comprise about 92% water, about 8% acetonitrile, methanol, or a mixture thereof, and about 0.1% formic acid, acetic acid, or a mixture thereof. In some embodiments, Solvent A, as described herein, may comprise about 93% water, about 7% acetonitrile, methanol, or a mixture thereof, and about 0.1% formic acid, acetic acid, or a mixture thereof. In certain embodiments, Solvent A, as described herein, may comprise about 94% water, about 6% acetonitrile, methanol, or a mixture thereof, and about 0.1% formic acid, acetic acid, or a mixture thereof. In some embodiments, Solvent A, as described herein, may comprise about 95% water, about 5% acetonitrile, methanol, or a mixture thereof, and about 0.1% formic acid, acetic acid, or a mixture thereof. In certain embodiments, Solvent A, as described herein, may comprise about 96% water, about 4% acetonitrile, methanol, or a mixture thereof, and about 0.1% formic acid, acetic acid, or a mixture thereof. In some embodiments, Solvent A, as described herein, may comprise about 97% water, about 3% acetonitrile, methanol, or a mixture thereof, and about 0.1% formic acid, acetic acid, or a mixture thereof. In certain embodiments, Solvent A, as described herein, may comprise about 98% water, about 2% acetonitrile, methanol, or a mixture thereof, and about 0.1% formic acid, acetic acid, or a mixture thereof. In some embodiments, Solvent A, as described herein, may comprise about 99% water, about 1% acetonitrile, methanol, or a mixture thereof, and about 0.1% formic acid, acetic acid, or a mixture thereof. In certain embodiments, Solvent A, as described herein, may comprise about 100% water, about 0% acetonitrile, methanol, or a mixture thereof, and about 0.1% formic acid, acetic acid, or a mixture thereof.

In certain embodiments, Solvent B, as described herein, may comprise about 90% acetonitrile, methanol, or a mixture thereof, about 10% water, and about 0.1% formic acid, acetic acid, or a mixture thereof. In some embodiments, Solvent B, as described herein, may comprise about 91% acetonitrile, methanol, or a mixture thereof, about 9% water, and about 0.1% formic acid, acetic acid, or a mixture thereof. In certain embodiments, Solvent B, as described herein, may comprise about 92% acetonitrile, methanol, or a mixture thereof, about 8% water, and about 0.1% formic acid, acetic acid, or a mixture thereof. In some embodiments, Solvent B, as described herein, may comprise about 93% acetonitrile, methanol, or a mixture thereof, about 7% water, and about 0.1% formic acid, acetic acid, or a mixture thereof. In certain embodiments, Solvent B, as described herein, may comprise about 94% acetonitrile, methanol, or a mixture thereof, about 6% water, and about 0.1% formic acid, acetic acid, or a mixture thereof. In some embodiments, Solvent B, as described herein, may comprise about 95% acetonitrile, methanol, or a mixture thereof, about 5% water, and about 0.1% formic acid, acetic acid, or a mixture thereof. In certain embodiments, Solvent B, as described herein, may comprise about 96% acetonitrile, methanol, or a mixture thereof, about 4% water, and about 0.1% formic acid, acetic acid, or a mixture thereof. In some embodiments, Solvent B, as described herein, may comprise about 97% acetonitrile, methanol, or a mixture thereof, about 3% water, and about 0.1% formic acid, acetic acid, or a mixture thereof. In certain embodiments, Solvent B, as described herein, may comprise about 98% acetonitrile, methanol, or a mixture thereof, about 2% water, and about 0.1% formic acid, acetic acid, or a mixture thereof. In some embodiments, Solvent B, as described herein, may comprise about 99% acetonitrile, methanol, or a mixture thereof, about 1% water, and about 0.1% formic acid, acetic acid, or a mixture thereof. In certain embodiments, Solvent B, as described herein, may comprise about 100% acetonitrile, methanol, or a mixture thereof, about 0% water, and about 0.1% formic acid, acetic acid, or a mixture thereof.

In some embodiments, a Gradient Method, as described herein, may specify a composition of a mobile phase, as described herein, as a percentage by volume of Solvent A and Solvent B, that is different at two or more time points defining a run time, as described herein.

In certain embodiments, a Gradient Method, as described herein, may specify a composition of a mobile phase, as described herein, for a run time of at least 5 minutes, wherein: (i) at the start of the run time the composition of the mobile phase may be about 90% Solvent A (10% Solvent B) to about 100% Solvent A (0% Solvent B), as described herein; (ii) for about 4% to about 8% of the total run time, the composition of the mobile phase may be constant at about 90% Solvent A (10% Solvent B) to about 100% Solvent A (0% Solvent B), as described herein; (iii) after step (ii), for about 6% to about 10% of the run total run time, the composition of the mobile phase may become about 70% Solvent A (30% Solvent B) to about 90% Solvent A (10% Solvent B), as described herein; (iv) after step (iii), for about 40% to about 60% of the run total run time, the composition of the mobile phase may be constant at about 70% Solvent A (30% Solvent B) to about 90% Solvent A (10% Solvent B), as described herein; (v) after step (iv), for about 6% to about 10% of the total run time, the composition of the mobile phase may become about 0% Solvent A (100% Solvent B) to about 10% Solvent A (90% Solvent B), as described herein; (vi) after step (v), for about 12% to about 16% of the total run time, the composition of the mobile phase may be constant at about 0% Solvent A (100% Solvent B) to about 10% Solvent A (90% Solvent B), as described herein; (vii) after step (vi), the composition of the mobile phase may become about 90% Solvent A (10% Solvent B) to about 100% Solvent A (0% Solvent B), as described herein; and (viii) after step (vii), for about 12% to about 16% of the total run time, the composition of the mobile phase may be constant at about 90% Solvent A (10% Solvent B) to about 100% Solvent A (0% Solvent B), as described herein.

In some embodiments, a Gradient Method, as described herein, may specify a composition of a mobile phase, as described herein, for a run time of at least 5 minutes, wherein: (i) at the start of the run time the composition of the mobile phase may be about 90% Solvent A (10% Solvent B) to about 100% Solvent A (0% Solvent B), as described herein; (ii) for about 2% to about 10% of the total run time, the composition of the mobile phase may be constant at about 90% Solvent A (10% Solvent B) to about 100% Solvent A (0% Solvent B), as described herein; (iii) after step (ii), for about 4% to about 12% of the run total run time, the composition of the mobile phase may become about 70% Solvent A (30% Solvent B) to about 90% Solvent A (10% Solvent B), as described herein; (iv) after step (iii), for about 30% to about 70% of the run total run time, the composition of the mobile phase may be constant at about 70% Solvent A (30% Solvent B) to about 90% Solvent A (10% Solvent B), as described herein; (v) after step (iv), for about 4% to about 12% of the total run time, the composition of the mobile phase may become about 0% Solvent A (100% Solvent B) to about 10% Solvent A (90% Solvent B), as described herein; (vi) after step (v), for about 10% to about 18% of the total run time, the composition of the mobile phase may be constant at about 0% Solvent A (100% Solvent B) to about 10% Solvent A (90% Solvent B), as described herein; (vii) after step (vi), the composition of the mobile phase may become about 90% Solvent A (10% Solvent B) to about 100% Solvent A (0% Solvent B), as described herein; (viii) after step (vii), for about 10% to about 18% of the total run time, the composition of the mobile phase may be constant at about 90% Solvent A (10% Solvent B) to about 100% Solvent A (0% Solvent B), as described herein.

In certain embodiments, a Gradient Method, as described herein, may specify a composition of a mobile phase, as described herein, for a run time of about 10 minutes, wherein: (i) at 0 minutes the composition of the mobile phase may be about 90% Solvent A (10% Solvent B) to about 100% Solvent A (0% Solvent B), as described herein; (ii) from about 0 minutes to about 0.6 minutes, the composition of the mobile phase may be constant at about 90% Solvent A (10% Solvent B) to about 100% Solvent A (0% Solvent B), as described herein; (iii) from about 0.6 minutes to about 1.4 minutes the composition of the mobile phase may become about 70% Solvent A (30% Solvent B) to about 90% Solvent A (10% Solvent B), as described herein; (iv) from about 1.4 minutes to about 6.4 minutes, the composition of the mobile phase may be constant at about 70% Solvent A (30% Solvent B) to about 90% Solvent A (10% Solvent B), as described herein; (v) from about 6.4 minutes to about 7.2 minutes, the composition of the mobile phase may become about 0% Solvent A (100% Solvent B) to about 10% Solvent A (90% Solvent B), as described herein; (vi) from about 7.2 minutes to about 8.6 minutes, the composition of the mobile phase may be constant at about 0% Solvent A (100% Solvent B) to about 10% Solvent A (90% Solvent B), as described herein; (vii) at about 8.6 minutes, the composition of the mobile phase may become about 90% Solvent A (10% Solvent B) to about 100% Solvent A (0% Solvent B), as described herein; (viii) from about 8.6 minutes to about 10 minutes, the composition of the mobile phase may be constant at about 90% Solvent A (10% Solvent B) to about 100% Solvent A (0% Solvent B), as described herein.

In some embodiments, a Gradient Method, as described herein, may specify a composition of a mobile phase, as described herein, for a run time of about 13 minutes, wherein: (i) at 0 minutes the composition of the mobile phase may be about 90% Solvent A (10% Solvent B) to about 100% Solvent A (0% Solvent B), as described herein; (ii) from about 0 minutes to about 0.7 minutes, the composition of the mobile phase may be constant at about 90% Solvent A (10% Solvent B) to about 100% Solvent A (0% Solvent B), as described herein; (iii) from about 0.7 minutes to about 1.8 minutes the composition of the mobile phase may become about 70% Solvent A (30% Solvent B) to about 90% Solvent A (10% Solvent B), as described herein; (iv) from about 1.8 minutes to about 8.3 minutes, the composition of the mobile phase may be constant at about 70% Solvent A (30% Solvent B) to about 90% Solvent A (10% Solvent B), as described herein; (v) from about 8.3 minutes to about 9.4 minutes, the composition of the mobile phase may become about 0% Solvent A (100% Solvent B) to about 10% Solvent A (90% Solvent B), as described herein; (vi) from about 9.4 minutes to about 11.2 minutes, the composition of the mobile phase may be constant at about 0% Solvent A (100% Solvent B) to about 10% Solvent A (90% Solvent B), as described herein; (vii) at about 11.2 minutes, the composition of the mobile phase may become about 90% Solvent A (10% Solvent B) to about 100% Solvent A (0% Solvent B), as described herein; (viii) from about 11.2 minutes to about 13 minutes, the composition of the mobile phase may be constant at about 90% Solvent A (10% Solvent B) to about 100% Solvent A (0% Solvent B), as described herein.

In certain embodiments, a Gradient Method, as described herein, may specify a composition of a mobile phase, as described herein, for a run time of about 15 minutes, wherein: (i) at 0 minutes the composition of the mobile phase may be about 90% Solvent A (10% Solvent B) to about 100% Solvent A (0% Solvent B), as described herein; (ii) from about 0 minutes to about 0.8 minutes, the composition of the mobile phase may be constant at about 90% Solvent A (10% Solvent B) to about 100% Solvent A (0% Solvent B), as described herein; (iii) from about 0.8 minutes to about 2.1 minutes the composition of the mobile phase may become about 70% Solvent A (30% Solvent B) to about 90% Solvent A (10% Solvent B), as described herein; (iv) from about 2.1 minutes to about 9.6 minutes, the composition of the mobile phase may be constant at about 70% Solvent A (30% Solvent B) to about 90% Solvent A (10% Solvent B), as described herein; (v) from about 9.6 minutes to about 10.8 minutes, the composition of the mobile phase may become about 0% Solvent A (100% Solvent B) to about 10% Solvent A (90% Solvent B), as described herein; (vi) from about 10.8 minutes to about 12.9 minutes, the composition of the mobile phase may be constant at about 0% Solvent A (100% Solvent B) to about 10% Solvent A (90% Solvent B), as described herein; (vii) at about 12.9 minutes, the composition of the mobile phase may become about 90% Solvent A (10% Solvent B) to about 100% Solvent A (0% Solvent B), as described herein; (viii) from about 12.9 minutes to about 15 minutes, the composition of the mobile phase may be constant at about 90% Solvent A (10% Solvent B) to about 100% Solvent A (0% Solvent B), as described herein.

In some embodiments, a Gradient Method, as described herein, may specify a composition of a mobile phase, as described herein, for a run time of about 18 minutes, wherein: (i) at 0 minutes the composition of the mobile phase may be about 90% Solvent A (10% Solvent B) to about 100% Solvent A (0% Solvent B), as described herein; (ii) from about 0 minutes to about 1.0 minutes, the composition of the mobile phase may be constant at about 90% Solvent A (10% Solvent B) to about 100% Solvent A (0% Solvent B), as described herein; (iii) from about 1.0 minutes to about 2.5 minutes the composition of the mobile phase may become about 70% Solvent A (30% Solvent B) to about 90% Solvent A (10% Solvent B), as described herein; (iv) from about 2.5 minutes to about 11.5 minutes, the composition of the mobile phase may be constant at about 70% Solvent A (30% Solvent B) to about 90% Solvent A (10% Solvent B), as described herein; (v) from about 11.5 minutes to about 13.0 minutes, the composition of the mobile phase may become about 0% Solvent A (100% Solvent B) to about 10% Solvent A (90% Solvent B), as described herein; (vi) from about 13.0 minutes to about 15.5 minutes, the composition of the mobile phase may be constant at about 0% Solvent A (100% Solvent B) to about 10% Solvent A (90% Solvent B), as described herein; (vii) at about 15.5 minutes, the composition of the mobile phase may become about 90% Solvent A (10% Solvent B) to about 100% Solvent A (0% Solvent B), as described herein; (viii) from about 15.5 minutes to about 18 minutes, the composition of the mobile phase may be constant at about 90% Solvent A (10% Solvent B) to about 100% Solvent A (0% Solvent B), as described herein.

In certain embodiments, a Gradient Method, as described herein, may specify a composition of a mobile phase, as described herein, for a run time of about 20 minutes, wherein: (i) at 0 minutes the composition of the mobile phase may be about 90% Solvent A (10% Solvent B) to about 100% Solvent A (0% Solvent B), as described herein; (ii) from about 0 minutes to about 1.1 minutes, the composition of the mobile phase may be constant at about 90% Solvent A (10% Solvent B) to about 100% Solvent A (0% Solvent B), as described herein; (iii) from about 1.1 minutes to about 2.8 minutes the composition of the mobile phase may become about 70% Solvent A (30% Solvent B) to about 90% Solvent A (10% Solvent B), as described herein; (iv) from about 2.8 minutes to about 12.8 minutes, the composition of the mobile phase may be constant at about 70% Solvent A (30% Solvent B) to about 90% Solvent A (10% Solvent B), as described herein; (v) from about 12.8 minutes to about 14.4 minutes, the composition of the mobile phase may become about 0% Solvent A (100% Solvent B) to about 10% Solvent A (90% Solvent B), as described herein; (vi) from about 14.4 minutes to about 17.2 minutes, the composition of the mobile phase may be constant at about 0% Solvent A (100% Solvent B) to about 10% Solvent A (90% Solvent B), as described herein; (vii) at about 17.2 minutes, the composition of the mobile phase may become about 90% Solvent A (10% Solvent B) to about 100% Solvent A (0% Solvent B), as described herein; (viii) from about 17.2 minutes to about 20 minutes, the composition of the mobile phase may be constant at about 90% Solvent A (10% Solvent B) to about 100% Solvent A (0% Solvent B), as described herein.

In some embodiments, a Gradient Method, as described herein, may specify a composition of a mobile phase, as described herein, for a run time of about 22 minutes, wherein: (i) at 0 minutes the composition of the mobile phase may be about 90% Solvent A (10% Solvent B) to about 100% Solvent A (0% Solvent B), as described herein; (ii) from about 0 minutes to about 1.2 minutes, the composition of the mobile phase may be constant at about 90% Solvent A (10% Solvent B) to about 100% Solvent A (0% Solvent B), as described herein; (iii) from about 1.2 minutes to about 3.1 minutes the composition of the mobile phase may become about 70% Solvent A (30% Solvent B) to about 90% Solvent A (10% Solvent B), as described herein; (iv) from about 3.1 minutes to about 14.1 minutes, the composition of the mobile phase may be constant at about 70% Solvent A (30% Solvent B) to about 90% Solvent A (10% Solvent B), as described herein; (v) from about 14.1 minutes to about 15.9 minutes, the composition of the mobile phase may become about 0% Solvent A (100% Solvent B) to about 10% Solvent A (90% Solvent B), as described herein; (vi) from about 15.9 minutes to about 18.9 minutes, the composition of the mobile phase may be constant at about 0% Solvent A (100% Solvent B) to about 10% Solvent A (90% Solvent B), as described herein; (vii) at about 18.9 minutes, the composition of the mobile phase may become about 90% Solvent A (10% Solvent B) to about 100% Solvent A (0% Solvent B), as described herein; (viii) from about 18.9 minutes to about 22 minutes, the composition of the mobile phase may be constant at about 90% Solvent A (10% Solvent B) to about 100% Solvent A (0% Solvent B), as described herein.

In certain embodiments, a Gradient Method, as described herein, may specify a composition of a mobile phase, as described herein, for a run time of about 25 minutes, wherein: (i) at 0 minutes the composition of the mobile phase may be about 90% Solvent A (10% Solvent B) to about 100% Solvent A (0% Solvent B), as described herein; (ii) from about 0 minutes to about 1.4 minutes, the composition of the mobile phase may be constant at about 90% Solvent A (10% Solvent B) to about 100% Solvent A (0% Solvent B), as described herein; (iii) from about 1.4 minutes to about 3.5 minutes the composition of the mobile phase may become about 70% Solvent A (30% Solvent B) to about 90% Solvent A (10% Solvent B), as described herein; (iv) from about 3.5 minutes to about 16.0 minutes, the composition of the mobile phase may be constant at about 70% Solvent A (30% Solvent B) to about 90% Solvent A (10% Solvent B), as described herein; (v) from about 16.0 minutes to about 18.1 minutes, the composition of the mobile phase may become about 0% Solvent A (100% Solvent B) to about 10% Solvent A (90% Solvent B), as described herein; (vi) from about 18.1 minutes to about 21.5 minutes, the composition of the mobile phase may be constant at about 0% Solvent A (100% Solvent B) to about 10% Solvent A (90% Solvent B), as described herein; (vii) at about 21.5 minutes, the composition of the mobile phase may become about 90% Solvent A (10% Solvent B) to about 100% Solvent A (0% Solvent B), as described herein; (viii) from about 21.5 minutes to about 25 minutes, the composition of the mobile phase may be constant at about 90% Solvent A (10% Solvent B) to about 100% Solvent A (0% Solvent B), as described herein.

In some embodiments, a Gradient Method, as described herein, may provide a separation factor between sucralose-6-acetate and at least one other compound in a sucralose-containing material, as described herein, including, but not limited to sucralose, dextrose, maltodextrin, dextrin, as well as any other excipient or additive that would commonly be found in artificial sweeteners, food additives, foods, drinks/beverages, functional foods, nutritional products, supplements, nootropics, nutraceuticals, and/or medicines. In certain embodiments, a Gradient Method, as described herein, may preferably provide a separation factor between sucralose-6-acetate and another compound in a sucralose-containing material, as described herein, including, but not limited to sucralose, dextrose, maltodextrin, dextrin, as well as any other excipient or additive that would commonly be found in artificial sweeteners, food additives, foods, drinks/beverages, and/or functional foods. As used herein, the term, “separation factor” refers to the difference in retention time between sucralose-6-acetate and at least one other compound in a sucralose-containing material, as described herein, when a Gradient Method, as described herein, is implemented on an HPLC Unit and/or a UHPLC Unit, as described herein, for a sample comprising a sucralose-containing material, as described herein. In certain embodiments, a Gradient Method, as described herein, may provide a separation factor between sucralose-6-acetate and at least one other compound in a sucralose-containing material, as described herein, of at least 0.5 minutes, at least 0.6 minutes, at least 0.7 minutes, at least 0.8 minutes, at least 0.9 minutes, at least 1.0 minute, at least 1.1 minutes, at least 1.2 minutes, at least 1.3 minutes, at least 1.4 minutes, at least 1.5 minutes, at least 1.6 minutes, at least 1.7 minutes, 1.8 minutes, at least 1.9 minutes, at least 2.0 minutes, at least 2.1 minutes, at least 2.2 minutes, at least 2.3 minutes, at least 2.4 minutes, at least 2.5 minutes, at least 2.6 minutes, at least 2.7 minutes, at least 2.8 minutes, at least 2.9 minutes, at least 3.0 minutes, at least 3.1 minutes, at least 3.2 minutes, at least 3.3 minutes, at least 3.4 minutes, at least 3.5 minutes, at least 3.6 minutes, at least 3.7 minutes, at least 3.8 minutes, at least 3.9 minutes, at least 4.0 minutes, at least 4.1 minutes, at least 4.2 minutes, at least 4.3 minutes, at least 4.4 minutes, at least 4.5 minutes, at least 4.6 minutes, at least 4.7 minutes, at least 4.8 minutes, at least 4.9 minutes, at least 5.0 minutes, or any fraction or integer in between any two of the preceding amounts.

In certain embodiments, an Isocratic Method and/or a Gradient Method, as described herein, as described herein, may be configured to operate in compliance with standards set by one or more regulatory agencies. In certain embodiments, a regulatory agency, as described herein, may refer to the European Food Safety Authority (EFSA), the Environmental Protection Agency (EPA), the Food and Drug Administration (FDA), any combination of the foregoing, and the like. In some embodiments, an Isocratic Method and/or a Gradient Method, as described herein, may be configured to operate in compliance with the Good Laboratory Practice for Nonclinical Laboratory Studies, as that term is used in 21 C.F.R. § 58 et seq. In certain embodiments, an Isocratic Method and/or a Gradient Method, as described herein, may be configured to operate in compliance with the Quality Guidelines, as that term is used by the International Conference on Harmonization (ICH).

Some embodiments provide methods for preparing samples configured to be analyzed with the analytical method for quantitatively determining an amount of sucralose-6-acetate in a sucralose-containing material, described herein. In some embodiments, methods for preparing samples, as described herein, may define preparation of one or more of the following: (i) one or more sucralose-containing material samples; (ii) one or more calibration curve samples; and (iii) one or more validation samples.

In some embodiments, methods for preparing samples, as described herein, may define preparation of at least one calibration curve samples. In certain embodiments, calibration curve samples, as described herein, may be prepared by dissolving sucralose-6-acetate in an aqueous solvent, as described herein, and optionally containing an additive, as described herein, such that the calibration curve samples cover a range of concentrations of sucralose-6-acetate spanning about 0.1 ppb to about 100 ppm, preferably about 0.1 ppb to about 10 ppm, more preferably about 0.1 ppb to about 1 ppm, even more preferably about 0.1 ppb to about 100 ppb, or most preferably about 1 ppb to about 100 ppb. In certain embodiments, one or more calibration curve samples, as described herein, may be further comprise an internal standard dissolved therein, such that the concentration of the internal standard in the sample would be less than about 2 μg/mL, preferably less than about 1 μg/mL, more preferably less than about 500 ng/mL, even more preferably less than about 100 ng/mL, or most preferably less than about 20 ng/mL. As used herein, the term, “internal standard” generally refers to an organic compound that is soluble in the aqueous solvent used to dissolve the sucralose-containing material and has a similar retention time to sucralose-6-acetate. In certain embodiments, an internal standard, as described herein, may specifically refer to salicylic acid.

In certain embodiments, methods for preparing samples, as described herein, may define preparation of one or more validation samples. In certain embodiments, one or more validation samples, as described herein, may be prepared by dissolving sucralose-6-acetate in an aqueous solvent, as described herein, and optionally containing an additive, as described herein, such that the concentration of sucralose-6-acetate in the sample falls within a range of sucralose-6-acetate concentrations encompassed by calibration curve samples, as described herein. In some embodiments, for calibration curve samples, as described herein, covering range of sucralose-6-acetate concentrations spanning about 0.1 ppb to about 100 ppm, one or more validation samples may be prepared at concentrations of: (i) about 0.5 ppb to about 5 ppb; (ii) about 300 ppb to about 30 ppm; (iii) about 60 ppm to about 80 ppm; or (iv) any combination of the foregoing. In certain embodiments, for calibration curve samples, as described herein, covering range of sucralose-6-acetate concentrations spanning about 0.1 ppb to about 10 ppm, one or more validation samples may be prepared at concentrations of: (i) about 0.5 ppb to about 5 ppb; (ii) about 30 ppb to about 3 ppm; (iii) about 6 ppm to about 8 ppm; or (iv) any combination of the foregoing. In some embodiments, for calibration curve samples, as described herein, covering range of sucralose-6-acetate concentrations spanning about 0.1 ppb to about 1 ppm, one or more validation samples may be prepared at concentrations of: (i) about 0.5 ppb to about 5 ppb; (ii) about 30 ppb to about 300 ppb; (iii) about 600 ppb to about 800 ppb; or (iv) any combination of the foregoing. In certain embodiments, for calibration curve samples, as described herein, covering range of sucralose-6-acetate concentrations spanning about 0.1 ppb to about 100 ppb, one or more validation samples may be prepared at concentrations of: (i) about 0.5 ppb to about 5 ppb; (ii) about 30 ppb to about 60 ppb; (iii) about 70 ppb to about 80 ppb; or (iv) any combination of the foregoing. In some embodiments, for calibration curve samples, as described herein, covering range of sucralose-6-acetate concentrations spanning about 1 ppb to about 100 ppb, one or more validation samples may be prepared at concentrations of: (i) about 1 ppb to about 5 ppb; (ii) about 30 ppb to about 60 ppb; (iii) about 70 ppb to about 80 ppb; or (iv) any combination of the foregoing. In certain embodiments, one or more validation samples, as described herein, may be further comprise an internal standard, as described herein, dissolved therein, such that the concentration of the internal standard in the sample would be less than about 2 μg/mL, preferably less than about 1 μg/mL, more preferably less than about 500 ng/ml, even more preferably less than about 100 ng/mL, or most preferably less than about 20 ng/mL.

In certain embodiments, methods for preparing samples, as described herein, may define preparation of one or more sucralose-containing material samples. In some embodiments, one or more sucralose-containing material samples, as described herein, may be prepared by dissolving a sucralose-containing material in an aqueous solvent, as described herein, and optionally containing an additive, as described herein, such that the concentration of sucralose-6-acetate in the sample falls within a range of sucralose-6-acetate concentrations encompassed by calibration curve samples, as described herein. In certain embodiments, one or more validation samples, as described herein, may be further comprise an internal standard, as described herein, dissolved therein, such that the concentration of the internal standard in the sample would be less than about 2 μg/mL, preferably less than about 1 μg/mL, more preferably less than about 500 ng/mL, even more preferably less than about 100 ng/mL, or most preferably less than about 20 ng/mL.

In some embodiments, methods for preparing samples, as described herein, may be configured to operate in compliance with standards set by one or more regulatory agencies. In certain embodiments, a regulatory agency, as described herein, may refer to the European Food Safety Authority (EFSA), the Environmental Protection Agency (EPA), the Food and Drug Administration (FDA), any combination of the foregoing, and the like. In some embodiments, methods for preparing samples, as described herein, may be configured to operate in compliance with the Good Laboratory Practice for Nonclinical Laboratory Studies, as that term is used in 21 C.F.R. § 58 et seq. In certain embodiments, methods for preparing samples, as described herein, may be configured to operate in compliance with the Quality Guidelines, as that term is used by the International Conference on Harmonization (ICH).

In some embodiments, an analytical method for quantitatively determining an amount of sucralose-6-acetate in a sucralose-containing material, as described herein, may provide a mass spectrometer (“MS Detector”) provided downstream of a liquid chromatography separation unit, as described herein, and configured to perform mass spectrometry on an eluent comprising a sucralose-containing material from a liquid chromatography separation unit, as described herein. In certain embodiments, a liquid chromatography separation unit, as described herein, may be configured to divert a portion of an eluent, as described herein, such that the diverted portion of the eluent does not enter an MS Detector, as described herein. In some embodiments, a portion of an eluent, as described herein, may be diverted via a valve that can be provided between a liquid chromatography separation unit, as described herein, and an MS Detector, as described herein. In some embodiments, a diverted portion of the eluent, as described herein, may contain one or more compounds selected from the group consisting of sucralose, dextrose, maltodextrin, dextrin, as well as any other excipient or additive that would commonly be found in artificial sweeteners, food additives, foods, drinks/beverages, functional foods, nutritional products, supplements, nootropics, nutraceuticals, and/or medicines. In certain embodiments, diverted portion of the eluent, as described herein, may preferably contain one or more compounds selected from the group consisting of sucralose, dextrose, maltodextrin, dextrin, as well as any other excipient or additive that would commonly be found in artificial sweeteners, food additives, foods, drinks/beverages, and/or functional foods.

In some embodiments, a diverted portion of the eluent, as described herein, may be provided to a detection means, as described herein, to quantitatively determine an amount of one or more compounds that are not sucralose-6-acetate present in a diverted portion of the eluent, as described herein. In certain embodiments, a diverted portion of the eluent, as described herein, may preferably be provided to a charged aerosol detector, as described herein, to quantitatively determine an amount of one or more compounds that are not sucralose-6-acetate present in a diverted portion of the eluent, as described herein.

In some embodiments, an MS Detector, as described herein, may utilize one or more ionization means selected from the group consisting of electron impact ionization, fast atom bombardment, electrospray ionization, atmospheric pressure chemical ionization, matrix-assisted laser desorption ionization, plasma and glow discharge ionization, field ionization, laser ionization, plasma-desorption ionization, resonance ionization, secondary ionization, spark source ionization, thermal ionization, and any combination thereof. In certain embodiments, an MS Detector, as described herein, may preferably utilize electrospray ionization and atmospheric pressure chemical ionization as an ionization means, as described herein.

In some embodiments, an MS Detector, as described herein, may define one or more ionization conditions selected from the group consisting of an interface temperature, a desolvation line temperature, a heat block temperature, a drying gas, a flow rate of the drying gas, a heating gas, a flow rate of the heating gas, a nebulizer gas, a flow rate of the nebulizer gas, a collision gas, and any combination thereof.

In certain embodiments, an interface temperature, as described herein, may be about 100° C. to about 500° C.

In some embodiments, a desolvation line temperature, as described herein, may be about 100° C. to about 500° C.

In certain embodiments, a heat block temperature, as described herein may be about 100° C. to about 500° C.

In some embodiments, a drying gas, as described herein, may be nitrogen or dry air. In certain embodiments, a flow rate of the drying gas, as described herein, may be about 1 L/min to about 20 L/min.

In some embodiments, a heating gas, as described herein, may be nitrogen or dry air. In certain embodiments, a flow rate of the heating gas, as described herein, may be about 1 L/min to about 20 L/min.

In some embodiments, a nebulizer gas, as described herein, may be nitrogen or dry air. In certain embodiments, a flow rate of the nebulizer gas, as described herein, may be about 0.5 L/min to about 10 L/min.

In some embodiments, a collision gas, as described herein may be argon.

In certain embodiments, an MS Detector, as described herein, may be configured to operate in an ionization mode.

In some embodiments, an ionization mode, as described herein, may be a positive-ion mode.

In certain embodiments, when an MS Detector, as described herein, is configured to operate in a positive-ion mode, the MS Detector can determine (or provide to a liquid chromatography separation unit, as described herein, to determine) a total ion count for one or more positive-ion mode mass-to-charge ratios, to generate an MS⁺ chromatogram. As used herein, the term “MS⁺ chromatogram” refers to a visual representation, e.g., a graph, of a total ion count for one or more positive-ion mode mass-to-charge ratios as a function of time, wherein temporal changes in total ion count for one or more positive-ion mode mass-to-charge ratios data generate one or more peaks, each comprising a peak area.

In certain embodiments, a positive-ion mode mass-to-charge ratio, as described herein, may correspond to a positive ion characteristic of sucralose-6-acetate that is selected from the group consisting of [M+3H]3+, [M+2H+Na]3+, [M+H+2Na]3+, [M+3Na]3+, [M+2H]2+, [M+H+NH4]2+, [M+H+Na]2+, [M+H+K]2+, [M+ACN+2H]2+, [M+2Na]2+, [M+2ACN+2H]2+, [M+3ACN+2H]2+, [M+H]+, [M+NH4]+, [M+Na]+, [M+CH3OH+H]+, [M+K]+, [M+ACN+H]+, [M+2Na−H]+, [M+IsoProp+H]+, [M+ACN+Na]+, [M+2K−H]+, [M+DMSO+H]+, [M+2ACN+H]+, [M+IsoProp+Na+H]+, [2M+H]+, [2M+NH4]+, [2M+Na]+, [2M+K]+, [2M+ACN+H]+, [2M+ACN+Na]+, and any combination thereof. As referenced herein with respect to positive ions, as described herein, “M” refers to a monoisotopic mass of sucralose-6-acetate, “H” refers to a monoisotopic mass of hydrogen, “Na” refers to a monoisotopic mass of sodium, “NH4” refers to a monoisotopic mass of ammonium, “K” refers to a monoisotopic mass of potassium, “ACN” refers to a monoisotopic mass of acetonitrile, “CH3OH” refers to a monoisotopic mass of methanol, “IsoProp” refers to a monoisotopic mass of isopropanol, “DMSO” refers to a monoisotopic mass of dimethylsulfoxide, a number preceding any of the foregoing abbreviations refers to a multiple of that abbreviation, and “+,” “2+,” and “3+,” following a bracketed term refer to a charge of the ion.

In some embodiments, a positive-ion mode mass-to-charge ratio, as described herein, may correspond to a positive ion characteristic of a compound in the eluent that is not sucralose-6-acetate, as described herein, that is selected from the group consisting of [M+3H]3+, [M+2H+Na]3+, [M+H+2Na]3+, [M+3Na]3+, [M+2H]2+, [M+H+NH4]2+, [M+H+Na]2+, [M+H+K]2+, [M+ACN+2H]2+, [M+2Na]2+, [M+2ACN+2H]2+, [M+3ACN+2H]2+, [M+H]+, [M+NH4]+, [M+Na]+, [M+CH3OH+H]+, [M+K]+, [M+ACN+H]+, [M+2Na−H]+, [M+IsoProp+H]+, [M+ACN+Na]+, [M+2K−H]+, [M+DMSO+H]+, [M+2ACN+H]+, [M+IsoProp+Na+H]+, [2M+H]+, [2M+NH4]+, [2M+Na]+, [2M+K]+, [2M+ACN+H]+, [2M+ACN+Na]+, and any combination thereof. As referenced herein with respect to positive ions, as described herein, “M” refers to a monoisotopic mass of a compound in the eluent that is not sucralose-6-acetate, as described herein, “H” refers to a monoisotopic mass of hydrogen, “Na” refers to a monoisotopic mass of sodium, “NH4” refers to a monoisotopic mass of ammonium, “K” refers to a monoisotopic mass of potassium, “ACN” refers to a monoisotopic mass of acetonitrile, “CH3OH” refers to a monoisotopic mass of methanol, “IsoProp” refers to a monoisotopic mass of isopropanol, “DMSO” refers to a monoisotopic mass of dimethylsulfoxide, a number preceding any of the foregoing abbreviations refers to a multiple of that abbreviation, and “+,” “2+,” and “3+,” following a bracketed term refer to a charge of the ion.

In some embodiments, a positive ion characteristic of a compound in the eluent that is not sucralose-6-acetate, as described herein, may be a positive ion characteristic of one or more of sucralose, dextrose, and maltodextrin. In such embodiments, a positive ion, as described herein, may be selected from the group consisting of [M+3H]3+, [M+2H+Na]3+, [M+H+2Na]3+, [M+3Na]3+, [M+2H]2+, [M+H+NH4]2+, [M+H+Na]2+, [M+H+K]2+, [M+ACN+2H]2+, [M+2Na]2+, [M+2ACN+2H]2+, [M+3ACN+2H]2+, [M+H]+, [M+NH4]+, [M+Na]+, [M+CH3OH+H]+, [M+K]+, [M+ACN+H]+, [M+2Na−H]+, [M+IsoProp+H]+, [M+ACN+Na]+, [M+2K−H]+, [M+DMSO+H]+, [M+2ACN+H]+, [M+IsoProp+Na+H]+, [2M+H]+, [2M+NH4]+, [2M+Na]+, [2M+K]+, [2M+ACN+H]+, [2M+ACN+Na]+, and any combination thereof. As referenced herein with respect to positive ions, as described herein, “M” refers to a monoisotopic mass of sucralose, dextrose, or maltodextrin, “H” refers to a monoisotopic mass of hydrogen, “Na” refers to a monoisotopic mass of sodium, “NH4” refers to a monoisotopic mass of ammonium, “K” refers to a monoisotopic mass of potassium, “ACN” refers to a monoisotopic mass of acetonitrile, “CH3OH” refers to a monoisotopic mass of methanol, “IsoProp” refers to a monoisotopic mass of isopropanol, “DMSO” refers to a monoisotopic mass of dimethylsulfoxide, a number preceding any of the foregoing abbreviations refers to a multiple of that abbreviation, and “+,” “2+,” and “3+,” following a bracketed term refer to a charge of the ion.

In some embodiments, a positive-ion mode mass-to-charge ratio, as described herein, may correspond to a positive ion characteristic of salicylic acid, that is selected from the group consisting of [M+3H]3+, [M+2H+Na]3+, [M+H+2Na]3+, [M+3Na]3+, [M+2H]2+, [M+H+NH4]2+, [M+H+Na]2+, [M+H+K]2+, [M+ACN+2H]2+, [M+2Na]2+, [M+2ACN+2H]2+, [M+3ACN+2H]2+, [M+H]+, [M+NH4]+, [M+Na]+, [M+CH3OH+H]+, [M+K]+, [M+ACN+H]+, [M+2Na−H]+, [M+IsoProp+H]+, [M+ACN+Na]+, [M+2K−H]+, [M+DMSO+H]+, [M+2ACN+H]+, [M+IsoProp+Na+H]+, [2M+H]+, [2M+NH4]+, [2M+Na]+, [2M+K]+, [2M+ACN+H]+, [2M+ACN+Na]+, and any combination thereof. As referenced herein with respect to positive ions, as described herein, “M” refers to a monoisotopic mass of salicylic acid, “H” refers to a monoisotopic mass of hydrogen, “Na” refers to a monoisotopic mass of sodium, “NH4” refers to a monoisotopic mass of ammonium, “K” refers to a monoisotopic mass of potassium, “ACN” refers to a monoisotopic mass of acetonitrile, “CH3OH” refers to a monoisotopic mass of methanol, “IsoProp” refers to a monoisotopic mass of isopropanol, “DMSO” refers to a monoisotopic mass of dimethylsulfoxide, a number preceding any of the foregoing abbreviations refers to a multiple of that abbreviation, and “+,” “2+,” and “3+,” following a bracketed term refer to a charge of the ion.

In some embodiments, an ionization mode, as described herein, may be a negative-ion mode.

In certain embodiments, when an MS Detector, as described herein, is configured to operate in a negative-ion mode, the MS Detector can determine (or provide to a liquid chromatography separation unit, as described herein, to determine) a total ion count for one or more negative-ion mode mass-to-charge ratios, to generate an MS chromatogram. As used herein, the term “MS⁻ chromatogram” refers to a visual representation, e.g., a graph, of a total ion count for one or more negative-ion mode mass-to-charge ratios as a function of time, wherein temporal changes in total ion count for one or more negative-ion mode mass-to-charge ratios data generate one or more peaks, each comprising a peak area.

In some embodiments, a negative-ion mode mass-to-charge ratio, as described herein, may correspond to a negative ion characteristic of sucralose-6-acetate that is selected from the group consisting of [M−3H]3−, [M−2H]2−, [M−H2O−H]−, [M−H]−, [M+Na−2H]−, [M+Cl]−, [M+K−2H]−, [M+FA−H]−, [M+Hac−H]−, [M+Br]−, [M+TFA−H]−, [2M−H]−, [2M+FA−H]−, [2M+Hac−H]−, [3M−H]−, and any combination thereof. As referenced herein with respect to negative ions, as described herein, “M” refers to a monoisotopic mass of sucralose-6-acetate, “H” refers to a monoisotopic mass of hydrogen, “H2O” refers to a monoisotopic mass of water, “Na” refers to a monoisotopic mass of sodium, “Cl” refers to a monoisotopic mass of chlorine, “K” refers to a monoisotopic mass of potassium, “FA” refers to a monoisotopic mass of formic acid, “Hac” refers to a monoisotopic mass of acetic acid, “Br” refers to a monoisotopic mass of bromine, “TFA” refers to a monoisotopic mass of trifluoroacetic acid, a number preceding any of the foregoing abbreviations refers to a multiple of that abbreviation, and “−,” “2−,” and “3−,” following a bracketed term refer to refer to a charge of the ion.

In certain embodiments, a negative ion characteristic of sucralose-6-acetate may have a value for a negative-ion mode mass-to-charge ratio, as described herein, that is 437, 439, 473, 475, 477, 483, 485, 500, 502, 527, 529, 535, 537, 539, 589, 591, 593, 629, 631, 633, 747, 749, 854, 856, 858, 892, 894, or any combination thereof.

In some embodiments, a negative-ion mode mass-to-charge ratio, as described herein, may correspond to a negative ion characteristic of a compound in the eluent that is not sucralose-6-acetate, as described herein, that is selected from the group consisting of [M−3H]3−, [M−2H]2−, [M−H2O−H]−, [M−H]−, [M+Na−2H]−, [M+Cl]−, [M+K−2H]−, [M+FA−H]−, [M+Hac−H]−, [M+Br]−, [M+TFA−H]−, [2M−H]−, [2M+FA−H]−, [2M+Hac−H]−, [3M−H]−, and any combination thereof. As referenced herein with respect to negative ions, as described herein, “M” refers to a monoisotopic mass of a compound in the eluent that is not sucralose-6-acetate, as described herein, “H” refers to a monoisotopic mass of hydrogen, “H2O” refers to a monoisotopic mass of water, “Na” refers to a monoisotopic mass of sodium, “Cl” refers to a monoisotopic mass of chlorine, “K” refers to a monoisotopic mass of potassium, “FA” refers to a monoisotopic mass of formic acid, “Hac” refers to a monoisotopic mass of acetic acid, “Br” refers to a monoisotopic mass of bromine, “TFA” refers to a monoisotopic mass of trifluoroacetic acid, a number preceding any of the foregoing abbreviations refers to a multiple of that abbreviation, and “−,” “2−,” and “3−,” following a bracketed term refer to refer to a charge of the ion.

In some embodiments, a negative ion characteristic of a compound in the eluent that is not sucralose-6-acetate, as described herein, may be a negative ion characteristic of one or more of sucralose, dextrose, and maltodextrin. In such embodiments, a negative ion, as described herein, may be selected from the group consisting of [M−3H]3−, [M−2H]2−, [M−H2O−H]−, [M−H]−, [M+Na−2H]−, [M+C1]−, [M+K−2H]−, [M+FA−H]−, [M+Hac−H]−, [M+Br]−, [M+TFA−H]−, [2M−H]−, [2M+FA−H]−, [2M+Hac−H]−, [3M−H]−, and any combination thereof. As referenced herein with respect to negative ions, as described herein, “M” refers to a monoisotopic mass of sucralose, dextrose, or maltodextrin, “H” refers to a monoisotopic mass of hydrogen, “H2O” refers to a monoisotopic mass of water, “Na” refers to a monoisotopic mass of sodium, “Cl” refers to a monoisotopic mass of chlorine, “K” refers to a monoisotopic mass of potassium, “FA” refers to a monoisotopic mass of formic acid, “Hac” refers to a monoisotopic mass of acetic acid, “Br” refers to a monoisotopic mass of bromine, “TFA” refers to a monoisotopic mass of trifluoroacetic acid, a number preceding any of the foregoing abbreviations refers to a multiple of that abbreviation, and, “−”, “2−,” and “3−,” following a bracketed term refer to refer to a charge of the ion.

In certain embodiments, a negative ion characteristic of sucralose may have a value for a negative-ion mode mass-to-charge ratio, as described herein, that is 395, 397, or the combination thereof. In some embodiments, a negative ion characteristic of dextrose may have a value for a negative-ion mode mass-to-charge ratio, as described herein, that is 179.

In some embodiments, a negative-ion mode mass-to-charge ratio, as described herein, may correspond to a negative ion characteristic of salicylic acid, that is selected from the group consisting of [M−3H]3−, [M−2H]2−, [M−H2O−H]−, [M−H]−, [M+Na−2H]−, [M+C1]−, [M+K−2H]−, [M+FA−H]−, [M+Hac−H]−, [M+Br]−, [M+TFA−H]−, [2M−H]−, [2M+FA−H]−, [2M+Hac−H]−, [3M−H]−, and any combination thereof. As referenced herein with respect to negative ions, as described herein, “M” refers to a monoisotopic mass of salicylic acid, “H” refers to a monoisotopic mass of hydrogen, “H2O” refers to a monoisotopic mass of water, “Na” refers to a monoisotopic mass of sodium, “Cl” refers to a monoisotopic mass of chlorine, “K” refers to a monoisotopic mass of potassium, “FA” refers to a monoisotopic mass of formic acid, “Hac” refers to a monoisotopic mass of acetic acid, “Br” refers to a monoisotopic mass of bromine, “TFA” refers to a monoisotopic mass of trifluoroacetic acid, a number preceding any of the foregoing abbreviations refers to a multiple of that abbreviation, and “−,” “2−,” and “3−,” following a bracketed term refer to refer to a charge of the ion.

In certain embodiments, a negative ion characteristic of salicylic acid may have a value for a negative-ion mode mass-to-charge ratio, as described herein, that is 137.

In some embodiments, an ionization mode, as described herein, may be a positive-ion mode, as described herein, and a negative-ion mode, as described herein.

In certain embodiments, an MS Detector, as described herein, or a liquid chromatography separation unit, as described herein, may be configured to determine a peak area of all peaks present on an MS⁺ chromatogram and/or an MS⁻ chromatogram. In some embodiments, peak areas, as described herein, may be used to quantitatively determine: (i) an amount of sucralose-6-acetate in a sucralose-containing material, as described herein; (ii) an amount of one or more compounds that are not sucralose-6-acetate in a sucralose-containing material, as described herein; (iii) an amount of salicylic acid in a sample, as described herein; or (iv) any combination of the foregoing. The particular methods for quantitatively determining an amount of any of the foregoing are not particularly limited, and may be analogous to other quantitative methods disclosed herein-one such, non-limiting, method, may be, for example: (i) preparing calibration curve samples for one or more compound of interest, e.g., sucralose-6-acetate, sucralose, dextrose, maltodextrin, and/or salicylic acid, and one or more sucralose-containing material samples; (ii) determining a peak area for the compound of interest in each sample of the calibration curve samples at a specified positive-ion mass-to-charge ratio or a specified negative-ion mass-to-charge ratio; (iii) generating plotted data by plotting peak area of each calibration curve sample against its concentration; (iv) fitting the plotted data with a linear best-fit line; (v) determining a peak area of the compound of interest in each of the one or more sucralose-containing material samples containing compound of interest; (vi) calculating a concentration of the compound of interest in each of the one or more sucralose-containing material samples containing compound of interest from the linear best-fit line; and (vii) optionally, converting the concentration of the compound of interest in each of the one or more sucralose-containing material samples containing compound of interest to a mass or mass percent.

In certain embodiments, the foregoing methods using an MS Detector, as described herein, or a liquid chromatography separation unit, as described herein, may be used to quantitatively determine an amount of sucralose-6-acetate in a sucralose-containing material, as described herein, that is present at a concentration of about 0.1 ppb to about 10,000 ppm, preferably about 0.1 to about 1,000 ppm, more preferably about 0.1 to about 100 ppm, even more preferably about 0.1 to about 10 ppm, even more preferably about 0.1 to about 1 ppm, even more preferably about 0.1 to about 100 ppb, or most preferably about 1 to about 100 ppb.

In some embodiments, an analytical method for quantitatively determining an amount of sucralose-6-acetate in a sucralose-containing material, as described herein, may provide a tandem mass spectrometer (“MS/MS Detector”) provided downstream of a liquid chromatography separation unit, as described herein, and configured to perform tandem mass spectrometry on an eluent comprising a sucralose-containing material from a liquid chromatography separation unit, as described herein. In certain embodiments, a liquid chromatography separation unit, as described herein, may be configured to divert a portion of an eluent, as described herein, such that the diverted portion of the eluent does not enter an MS/MS Detector, as described herein. In some embodiments, a portion of an eluent, as described herein, may be diverted via a valve that can be provided between a liquid chromatography separation unit, as described herein, and an MS/MS Detector, as described herein. In some embodiments, a diverted portion of the eluent, as described herein, may contain one or more compounds selected from the group consisting of sucralose, dextrose, maltodextrin, dextrin, as well as any other excipient or additive that would commonly be found in artificial sweeteners, food additives, foods, drinks/beverages, functional foods, nutritional products, supplements, nootropics, nutraceuticals, and/or medicines. In certain embodiments, diverted portion of the eluent, as described herein, may preferably contain one or more compounds selected from the group consisting of sucralose, dextrose, maltodextrin, dextrin, as well as any other excipient or additive that would commonly be found in artificial sweeteners, food additives, foods, drinks/beverages, and/or functional foods.

In some embodiments, a diverted portion of the eluent, as described herein, may be provided to a detection means, as described herein, to quantitatively determine an amount of one or more compounds that are not sucralose-6-acetate present in a diverted portion of the eluent, as described herein. In certain embodiments, a diverted portion of the eluent, as described herein, may preferably be provided to a charged aerosol detector, as described herein, to quantitatively determine an amount of one or more compounds that are not sucralose-6-acetate present in a diverted portion of the eluent, as described herein.

In some embodiments, an MS/MS Detector, as described herein, may utilize one or more ionization means selected from the group consisting of electron impact ionization, fast atom bombardment, electrospray ionization, atmospheric pressure chemical ionization, matrix-assisted laser desorption ionization, plasma and glow discharge ionization, field ionization, laser ionization, plasma-desorption ionization, resonance ionization, secondary ionization, spark source ionization, thermal ionization, and any combination thereof. In certain embodiments, an MS/MS Detector, as described herein, may preferably utilize electrospray ionization and atmospheric pressure chemical ionization as an ionization means, as described herein.

In some embodiments, an MS/MS Detector, as described herein, may define one or more ionization conditions selected from the group consisting of an interface temperature, a desolvation line temperature, a heat block temperature, a drying gas, a flow rate of the drying gas, a heating gas, a flow rate of the heating gas, a nebulizer gas, a flow rate of the nebulizer gas, a collision gas, and any combination thereof.

In certain embodiments, an interface temperature, as described herein, may be about 100° C. to about 500° C.

In some embodiments, a desolvation line temperature, as described herein, may be about 100° C. to about 500° C.

In certain embodiments, a heat block temperature, as described herein may be about 100° C. to about 500° C.

In some embodiments, a drying gas, as described herein, may be nitrogen or dry air. In certain embodiments, a flow rate of the drying gas, as described herein, may be about 1 L/min to about 20 L/min.

In some embodiments, a heating gas, as described herein, may be nitrogen or dry air. In certain embodiments, a flow rate of the heating gas, as described herein, may be about 1 L/min to about 20 L/min.

In some embodiments, a nebulizer gas, as described herein, may be nitrogen or dry air. In certain embodiments, a flow rate of the nebulizer gas, as described herein, may be about 0.5 L/min to about 10 L/min.

In some embodiments, a collision gas, as described herein may be argon.

In certain embodiments, an MS/MS Detector, as described herein, may be configured to operate in an ionization mode.

In some embodiments, an ionization mode, as described herein, may be a positive-ion mode.

In certain embodiments, when an MS/MS Detector, as described herein, is configured to operate in a positive-ion mode, the MS/MS Detector can determine (or provide to a liquid chromatography separation unit, as described herein, to determine) a total ion count for one or more positive-ion mode mass-to-charge ratios, to generate an MS⁺ chromatogram, as described herein.

In some embodiments, when an MS/MS Detector, as described herein, is configured to operate in a positive-ion mode, the MS/MS Detector can determine (or provide to a liquid chromatography separation unit, as described herein, to determine) a total ion count for one or more positive-ion mode mass-to-charge ratio transitions defining an initial positive-ion mode mass-to-charge ratio and a positive-ion mode mass-to-charge ratio for a fragment thereof, to generate an MS/MS⁺ chromatogram. As used herein, the term “MS/MS⁺ chromatogram” refers to a visual representation, e.g., a graph, of a total ion count for one or more positive-ion mode mass-to-charge ratio transitions as a function of time, wherein temporal changes in total ion count for one or more positive-ion mode mass-to-charge ratio transitions data generate one or more peaks, each comprising a peak area.

In certain embodiments, a positive-ion mode mass-to-charge ratio and/or an initial positive-ion mode mass-to-charge ratio, as described herein, may correspond to a positive ion characteristic of sucralose-6-acetate that is selected from the group consisting of [M+3H]3+, [M+2H+Na]3+, [M+H+2Na]3+, [M+3Na]3+, [M+2H]2+, [M+H+NH4]2+, [M+H+Na]2+, [M+H+K]2+, [M+ACN+2H]2+, [M+2Na]2+, [M+2ACN+2H]2+, [M+3ACN+2H]2+, [M+H]+, [M+NH4]+, [M+Na]+, [M+CH3OH+H]+, [M+K]+, [M+ACN+H]+, [M+2Na−H]+, [M+IsoProp+H]+, [M+ACN+Na]+, [M+2K−H]+, [M+DMSO+H]+, [M+2ACN+H]+, [M+IsoProp+Na+H]+, [2M+H]+, [2M+NH4]+, [2M+Na]+, [2M+K]+, [2M+ACN+H]+, [2M+ACN+Na]+, and any combination thereof. As referenced herein with respect to positive ions, as described herein, “M” refers to a monoisotopic mass of sucralose-6-acetate, “H” refers to a monoisotopic mass of hydrogen, “Na” refers to a monoisotopic mass of sodium, “NH4” refers to a monoisotopic mass of ammonium, “K” refers to a monoisotopic mass of potassium, “ACN” refers to a monoisotopic mass of acetonitrile, “CH3OH” refers to a monoisotopic mass of methanol, “IsoProp” refers to a monoisotopic mass of isopropanol, “DMSO” refers to a monoisotopic mass of dimethylsulfoxide, a number preceding any of the foregoing abbreviations refers to a multiple of that abbreviation, and “+,” “2+,” and “3+,” following a bracketed term refer to a charge of the ion.

In some embodiments, a positive-ion mode mass-to-charge ratio and/or an initial positive-ion mode mass-to-charge ratio, as described herein, may correspond to a positive ion characteristic of a compound in the eluent that is not sucralose-6-acetate, as described herein, that is selected from the group consisting of [M+3H]3+, [M+2H+Na]3+, [M+H+2Na]3+, [M+3Na]3+, [M+2H]2+, [M+H+NH4]2+, [M+H+Na]2+, [M+H+K]2+, [M+ACN+2H]2+, [M+2Na]2+, [M+2ACN+2H]2+, [M+3ACN+2H]2+, [M+H]+, [M+NH4]+, [M+Na]+, [M+CH3OH+H]+, [M+K]+, [M+ACN+H]+, [M+2Na−H]+, [M+IsoProp+H]+, [M+ACN+Na]+, [M+2K−H]+, [M+DMSO+H]+, [M+2ACN+H]+, [M+IsoProp+Na+H]+, [2M+H]+, [2M+NH4]+, [2M+Na]+, [2M+K]+, [2M+ACN+H]+, [2M+ACN+Na]+, and any combination thereof. As referenced herein with respect to positive ions, as described herein, “M” refers to a monoisotopic mass of a compound in the eluent that is not sucralose-6-acetate, as described herein, “H” refers to a monoisotopic mass of hydrogen, “Na” refers to a monoisotopic mass of sodium, “NH4” refers to a monoisotopic mass of ammonium, “K” refers to a monoisotopic mass of potassium, “ACN” refers to a monoisotopic mass of acetonitrile, “CH3OH” refers to a monoisotopic mass of methanol, “IsoProp” refers to a monoisotopic mass of isopropanol, “DMSO” refers to a monoisotopic mass of dimethylsulfoxide, a number preceding any of the foregoing abbreviations refers to a multiple of that abbreviation, and “+,” “2+,” and “3+,” following a bracketed term refer to a charge of the ion.

In some embodiments, an initial positive ion characteristic of a compound in the eluent that is not sucralose-6-acetate, as described herein, may be a positive ion characteristic of one or more of sucralose, dextrose, and maltodextrin. In such embodiments, a positive ion, as described herein, may be selected from the group consisting of [M+3H]3+, [M+2H+Na]3+, [M+H+2Na]3+, [M+3Na]3+, [M+2H]2+, [M+H+NH4]2+, [M+H+Na]2+, [M+H+K]2+, [M+ACN+2H]2+, [M+2Na]2+, [M+2ACN+2H]2+, [M+3ACN+2H]2+, [M+H]+, [M+NH4]+, [M+Na]+, [M+CH3OH+H]+, [M+K]+, [M+ACN+H]+, [M+2Na−H]+, [M+IsoProp+H]+, [M+ACN+Na]+, [M+2K−H]+, [M+DMSO+H]+, [M+2ACN+H]+, [M+IsoProp+Na+H]+, [2M+H]+, [2M+NH4]+, [2M+Na]+, [2M+K]+, [2M+ACN+H]+, [2M+ACN+Na]+, and any combination thereof. As referenced herein with respect to positive ions, as described herein, “M” refers to a monoisotopic mass of sucralose, dextrose, or maltodextrin, “H” refers to a monoisotopic mass of hydrogen, “Na” refers to a monoisotopic mass of sodium, “NH4” refers to a monoisotopic mass of ammonium, “K” refers to a monoisotopic mass of potassium, “ACN” refers to a monoisotopic mass of acetonitrile, “CH3OH” refers to a monoisotopic mass of methanol, “IsoProp” refers to a monoisotopic mass of isopropanol, “DMSO” refers to a monoisotopic mass of dimethylsulfoxide, a number preceding any of the foregoing abbreviations refers to a multiple of that abbreviation, and “+,” “2+,” and “3+,” following a bracketed term refer to a charge of the ion.

In some embodiments, a positive-ion mode mass-to-charge ratio and/or an initial positive-ion mode mass-to-charge ratio, as described herein, may correspond to a positive ion characteristic of salicylic acid, that is selected from the group consisting of [M+3H]3+, [M+2H+Na]3+, [M+H+2Na]3+, [M+3Na]3+, [M+2H]2+, [M+H+NH4]2+, [M+H+Na]2+, [M+H+K]2+, [M+ACN+2H]2+, [M+2Na]2+, [M+2ACN+2H]2+, [M+3ACN+2H]2+, [M+H]+, [M+NH4]+, [M+Na]+, [M+CH3OH+H]+, [M+K]+, [M+ACN+H]+, [M+2Na−H]+, [M+IsoProp+H]+, [M+ACN+Na]+, [M+2K−H]+, [M+DMSO+H]+, [M+2ACN+H]+, [M+IsoProp+Na+H]+, [2M+H]+, [2M+NH4]+, [2M+Na]+, [2M+K]+, [2M+ACN+H]+, [2M+ACN+Na]+, and any combination thereof. As referenced herein with respect to positive ions, as described herein, “M” refers to a monoisotopic mass of salicylic acid, “H” refers to a monoisotopic mass of hydrogen, “Na” refers to a monoisotopic mass of sodium, “NH4” refers to a monoisotopic mass of ammonium, “K” refers to a monoisotopic mass of potassium, “ACN” refers to a monoisotopic mass of acetonitrile, “CH3OH” refers to a monoisotopic mass of methanol, “IsoProp” refers to a monoisotopic mass of isopropanol, “DMSO” refers to a monoisotopic mass of dimethylsulfoxide, a number preceding any of the foregoing abbreviations refers to a multiple of that abbreviation, and “+,” “2+,” and “3+,” following a bracketed term refer to a charge of the ion.

In some embodiments, an ionization mode, as described herein, may be a negative-ion mode.

In certain embodiments, when an MS/MS Detector, as described herein, is configured to operate in a negative-ion mode, the MS/MS Detector can determine (or provide to a liquid chromatography separation unit, as described herein, to determine) a total ion count for one or more negative-ion mode mass-to-charge ratios, to generate an MS⁻ chromatogram, as described herein.

In some embodiments, when an MS/MS Detector, as described herein, is configured to operate in a negative-ion mode, the MS/MS Detector can determine (or provide to a liquid chromatography separation unit, as described herein, to determine) a total ion count for one or more negative-ion mode mass-to-charge ratio transitions defining an initial negative-ion mode mass-to-charge ratio and a negative-ion mode mass-to-charge ratio for a fragment thereof, to generate an MS/MS⁻ chromatogram. As used herein, the term “MS/MS⁻ chromatogram” refers to a visual representation, e.g., a graph, of a total ion count for one or more negative-ion mode mass-to-charge ratio transitions as a function of time, wherein temporal changes in total ion count for one or more negative-ion mode mass-to-charge ratio transitions data generate one or more peaks, each comprising a peak area.

In some embodiments, a negative-ion mode mass-to-charge ratio and/or an initial negative-ion mode mass-to-charge ratio, as described herein, may correspond to a negative ion characteristic of sucralose-6-acetate that is selected from the group consisting of [M−3H]3−, [M−2H]2−, [M−H2O−H]−, [M−H]−, [M+Na−2H]−, [M+Cl]−, [M+K−2H]−, [M+FA−H]−, [M+Hac−H]−, [M+Br]−, [M+TFA−H]−, [2M−H]−, [2M+FA−H]−, [2M+Hac−H]−, [3M−H]−, and any combination thereof. As referenced herein with respect to negative ions, as described herein, “M” refers to a monoisotopic mass of sucralose-6-acetate, “H” refers to a monoisotopic mass of hydrogen, “H2O” refers to a monoisotopic mass of water, “Na” refers to a monoisotopic mass of sodium, “Cl” refers to a monoisotopic mass of chlorine, “K” refers to a monoisotopic mass of potassium, “FA” refers to a monoisotopic mass of formic acid, “Hac” refers to a monoisotopic mass of acetic acid, “Br” refers to a monoisotopic mass of bromine, “TFA” refers to a monoisotopic mass of trifluoroacetic acid, a number preceding any of the foregoing abbreviations refers to a multiple of that abbreviation, and “−,” “2−,” and “3−,” following a bracketed term refer to refer to a charge of the ion.

In certain embodiments, a negative ion characteristic of sucralose-6-acetate may have a value for a negative-ion mode mass-to-charge ratio and/or an initial negative-ion mode mass-to-charge ratio, as described herein, that is 437, 439, 473, 475, 477, 483, 485, 500, 502, 527, 529, 535, 537, 539, 589, 591, 593, 629, 631, 633, 747, 749, 854, 856, 858, 892, 894, or any combination thereof.

In some embodiments, a negative ion characteristic of sucralose-6-acetate, may have a value for a fragment of an initial negative-ion mode mass-to-charge ratio, as described herein, that is 437 or 439.

In certain embodiments, a negative-ion mode mass-to-charge ratio transition characteristic of sucralose-6-acetate may have a values for an initial negative-ion mode mass-to-charge ratio (represented by “I”) and a fragment thereof (represented by “F”), as described herein, that are 473 (I) to 437 (F), 475 (I) to 439 (F), 483 (I) to 437 (F), 485 (I) to 439 (F), and any combination thereof.

In some embodiments, a negative-ion mode mass-to-charge ratio and/or an initial negative-ion mode mass-to-charge ratio, as described herein, may correspond to a negative ion characteristic of a compound in the eluent that is not sucralose-6-acetate, as described herein, that is selected from the group consisting of [M−3H]3−, [M−2H]2−, [M−H2O−H]−, [M−H]−, [M+Na−2H]−, [M+C1]−, [M+K−2H]−, [M+FA-H]−, [M+Hac−H]−, [M+Br]−, [M+TFA−H]−, [2M−H]−, [2M+FA−H]−, [2M+Hac−H]−, [3M−H]−, and any combination thereof. As referenced herein with respect to negative ions, as described herein, “M” refers to a monoisotopic mass of a compound in the eluent that is not sucralose-6-acetate, as described herein, “H” refers to a monoisotopic mass of hydrogen, “H2O” refers to a monoisotopic mass of water, “Na” refers to a monoisotopic mass of sodium, “Cl” refers to a monoisotopic mass of chlorine, “K” refers to a monoisotopic mass of potassium, “FA” refers to a monoisotopic mass of formic acid, “Hac” refers to a monoisotopic mass of acetic acid, “Br” refers to a monoisotopic mass of bromine, “TFA” refers to a monoisotopic mass of trifluoroacetic acid, a number preceding any of the foregoing abbreviations refers to a multiple of that abbreviation, and “,” “2−,” and “3−,” following a bracketed term refer to refer to a charge of the ion.

In certain embodiments, a negative ion characteristic of a compound in the eluent that is not sucralose-6-acetate, as described herein, may be a negative ion characteristic of one or more of sucralose, dextrose, and maltodextrin. In such embodiments, a negative ion, as described herein, may be selected from the group consisting of [M−3H]3−, [M−2H]2−, [M−H2O−H]−, [M−H]−, [M+Na−2H]−, [M+C1]−, [M+K−2H]−, [M+FA−H]−, [M+Hac−H]−, [M+Br]−, [M+TFA−H]−, [2M−H]−, [2M+FA−H]−, [2M+Hac−H]−, [3M−H]−, and any combination thereof. As referenced herein with respect to negative ions, as described herein, “M” refers to a monoisotopic mass of sucralose, dextrose, or maltodextrin, “H” refers to a monoisotopic mass of hydrogen, “H2O” refers to a monoisotopic mass of water, “Na” refers to a monoisotopic mass of sodium, “Cl” refers to a monoisotopic mass of chlorine, “K” refers to a monoisotopic mass of potassium, “FA” refers to a monoisotopic mass of formic acid, “Hac” refers to a monoisotopic mass of acetic acid, “Br” refers to a monoisotopic mass of bromine, “TFA” refers to a monoisotopic mass of trifluoroacetic acid, a number preceding any of the foregoing abbreviations refers to a multiple of that abbreviation, and “−,” “2−,” and “3−,” following a bracketed term refer to refer to a charge of the ion.

In some embodiments, a negative ion characteristic of sucralose may have a value for a negative-ion mode mass-to-charge ratio and/or an initial negative-ion mode mass-to-charge ratio, as described herein, that is 395, 397, or the combination thereof. In certain embodiments, a negative ion characteristic of dextrose may have a value for a negative-ion mode mass-to-charge ratio and/or an initial negative-ion mode mass-to-charge ratio, as described herein, that is 179.

In some embodiments, a negative-ion mode mass-to-charge ratio and/or an initial negative-ion mode mass-to-charge ratio, as described herein, may correspond to a negative ion characteristic of salicylic acid, that is selected from the group consisting of [M−3H]3−, [M−2H]2−, [M−H2O−H]−, [M−H]−, [M+Na−2H]−, [M+Cl]−, [M+K−2H]−, [M+FA−H]−, [M+Hac−H]−, [M+Br]−, [M+TFA−H]−, [2M−H]−, [2M+FA−H]−, [2M+Hac−H]−, [3M−H]−, and any combination thereof. As referenced herein with respect to negative ions, as described herein, “M” refers to a monoisotopic mass of salicylic acid, “H” refers to a monoisotopic mass of hydrogen, “H2O” refers to a monoisotopic mass of water, “Na” refers to a monoisotopic mass of sodium, “Cl” refers to a monoisotopic mass of chlorine, “K” refers to a monoisotopic mass of potassium, “FA” refers to a monoisotopic mass of formic acid, “Hac” refers to a monoisotopic mass of acetic acid, “Br” refers to a monoisotopic mass of bromine, “TFA” refers to a monoisotopic mass of trifluoroacetic acid, a number preceding any of the foregoing abbreviations refers to a multiple of that abbreviation, and “−,” “2−,” and “3−,” following a bracketed term refer to refer to a charge of the ion.

In certain embodiments, a negative ion characteristic of salicylic acid may have a value for a negative-ion mode mass-to-charge ratio and/or an initial negative-ion mode mass-to-charge ratio, as described herein, that is 137.

In some embodiments, a negative ion characteristic of salicylic acid may have a value for a fragment of an initial negative-ion mode mass-to-charge ratio, as described herein, that is 65, 75, or 93.

In certain embodiments, a negative-ion mode mass-to-charge ratio transition characteristic of salicylic acid may have a values for an initial negative-ion mode mass-to-charge ratio (represented by “I”) and a fragment thereof (represented by “F”), as described herein, that are 137 (I) to 65 (F), 137 (I) to 75 (F), 137 (I) to 93 (F), and any combination thereof.

In some embodiments, an ionization mode, as described herein, may be a positive-ion mode, as described herein, and a negative-ion mode, as described herein.

In certain embodiments, an MS/MS Detector, as described herein, or a liquid chromatography separation unit, as described herein, may be configured to determine a peak area of all peaks present on an MS/MS⁺ chromatogram and/or an MS/MS⁻ chromatogram. In some embodiments, peak areas, as described herein, may be used to quantitatively determine: (i) an amount of sucralose-6-acetate in a sucralose-containing material, as described herein; (ii) an amount of one or more compounds that are not sucralose-6-acetate in a sucralose-containing material, as described herein; (iii) an amount of salicylic acid in a sample, as described herein; or (iv) any combination of the foregoing. The particular methods for quantitatively determining an amount of any of the foregoing are not particularly limited, and may be analogous to other quantitative methods disclosed herein-one such, non-limiting, method, may be, for example: (i) preparing calibration curve samples for one or more compound of interest, e.g., sucralose-6-acetate, sucralose, dextrose, maltodextrin, and/or salicylic acid, and one or more sucralose-containing material samples; (ii) determining a peak area for the compound of interest in each sample of the calibration curve samples at a specified positive-ion mass-to-charge ratio transition or a specified negative-ion mass-to-charge ratio transition; (iii) generating plotted data by plotting peak area of each calibration curve sample against its concentration; (iv) fitting the plotted data with a linear best-fit line; (v) determining a peak area of the compound of interest in each of the one or more sucralose-containing material samples containing compound of interest; (vi) calculating a concentration of the compound of interest in each of the one or more sucralose-containing material samples containing compound of interest from the linear best-fit line; and (vii) optionally, converting the concentration of the compound of interest in each of the one or more sucralose-containing material samples containing compound of interest to a mass or mass percent.

In certain embodiments, the foregoing methods using an MS/MS Detector, as described herein, or a liquid chromatography separation unit, as described herein, may be used to quantitatively determine an amount of sucralose-6-acetate in a sucralose-containing material, as described herein, that is present at a concentration of about 0.1 ppb to about 10,000 ppm, preferably about 0.1 to about 1,000 ppm, more preferably about 0.1 to about 100 ppm, even more preferably about 0.1 to about 10 ppm, even more preferably about 0.1 to about 1 ppm, even more preferably about 0.1 to about 100 ppb, or most preferably about 1 to about 100 ppb.

In certain embodiments, an MS Detector and/or an MS/MS Detector, as described herein, as described herein, may be configured to operate in compliance with standards set by one or more regulatory agencies. In certain embodiments, a regulatory agency, as described herein, may refer to the European Food Safety Authority (EFSA), the Environmental Protection Agency (EPA), the Food and Drug Administration (FDA), any combination of the foregoing, and the like. In some embodiments, an MS Detector and/or an MS/MS Detector, as described herein, may be configured to operate in compliance with the Good Laboratory Practice for Nonclinical Laboratory Studies, as that term is used in 21 C.F.R. § 58 et seq. In certain embodiments, an MS Detector and/or an MS/MS Detector, as described herein, may be configured to operate in compliance with the Quality Guidelines, as that term is used by the International Conference on Harmonization (ICH).

Some embodiments provide methods for quantitatively determining an amount of sucralose-6-acetate in a sucralose-containing material for samples that have been analyzed with the methods discussed herein. In some embodiments, methods for quantitating, as described herein, may define one or more of the following: (i) standard curve generation; (ii) standard curve validation; and (iii) sucralose-6-acetate calculation.

In some embodiments, a method for generating a standard curve, as described herein, may define: (i) preparing sucralose-6-acetate calibration curve samples with the methods described herein; (ii) analyzing sucralose-6-acetate calibration curve samples with the methods described herein; (iii) determining a peak area for each sample of the sucralose-6-acetate calibration curve samples, as described herein; (iv) generating plotted data by plotting the peak area of each sucralose-6-acetate calibration curve sample against its concentration, as described herein; and (v) fitting the plotted data with a linear best-fit line, as described herein.

In certain embodiments, a method for validating a standard curve, as described herein, may define: (i) preparing sucralose-6-acetate validation samples with the methods described herein; (ii) analyzing sucralose-6-acetate validation samples with the methods described herein; (iii) determining a peak area for each sample of the sucralose-6-acetate validation samples, as described herein; (iv) calculating a concentration of sucralose-6-acetate in the validation samples from a linear best-fit line, as described herein; (v) determining that the calculated concentrations of sucralose-6-acetate in the validation samples comply with the following criteria (a) at least 67% of all validation samples are within 15% of their nominal value and (b) at least 50% of validation samples at a level, e.g., O (1 ppb), O (10 ppb), and the like, are within 15% of their nominal value; and optionally (vi) determining a limit of detection (“LOD”) for the method with LOD=3×SD (for at least 5 validation samples), wherein “SD” is the standard deviation of the at least 5 validation samples.

In some embodiments, a method for calculating sucralose-6-acetate, as described herein, may define: (i) preparing sucralose-containing material samples with the methods described herein; (ii) analyzing sucralose-containing material samples with the methods described herein; (iii) determining a peak area for each sample of the sucralose-containing material samples, as described herein; (iv) calculating a concentration of sucralose-6-acetate in the sucralose-containing material samples from a linear best-fit line, as described herein; and optionally (v) converting the concentration of sucralose-6-acetate in the sucralose-containing material samples to a mass percentage given by:

$${C}_{S 6 A} = \frac{{V}_{F} \times {D}_{F} \times {C}_{A}}{{W}_{S}} \qquad Equation ( 1 )$$

wherein, “C_{S6A}” refers to the mass percentage of sucralose-6-acetate in a sample in ng/g, “V_{F}” refers to the final volume of solution in mL, “D_{F}” refers to the dilution factor, “C_{A}” refers to the sucralose-6-acetate concentration determined in step (iv) in ng/mL, and “W_{S}” refers to the weight of the sample in g. The units disclosed herein are not particularly limited and may be adapted for different units—in such instances, dimensional analysis can be employed to ensure proper conversation from the equation provided above.

The following exemplary embodiments are provided as illustrations of various aspects of the disclosure provided herein and are not to be construed as limiting the scope of the disclosure contained herein in any way.

### EXAMPLES

### Example 1

Ten commercially-available sucralose-containing samples were obtained from various sources. Among these sucralose-containing samples, five samples were powders, three samples were liquids, one sample was a granular solid, and one sample was a tablet (“mini-tablet”). The details of the ten commercially-available products are provided below in Table 1.

TABLE 1

| Sample | | |
|---|---|---|
| Name | Product | Product Information |
| Powder 1 | Splenda ® Packet | UPC Code: 7 22776 20002 5 |
| Granular | Splenda ® Granular | UPC Code: 7 22776 21004 8 |
| | Sweetener | |
| Mini-tablet | Splenda ® Tablets | UPC Code: 7 22776 00053 3 |
| Powder 2 | Splenda ® Packet | UPC Code: 7 22776 20002 5 |
| Powder 3 | Splenda ® Packet | — |
| Powder 4 | Splenda ® Packet | UPC Code: 7 22776 00150 9 |
| Powder 5 | Splenda ® Packet | UPC Code: 7 22776 20041 4 |
| Liquid 1 | Yellow Splenda ® Zero | UPC Code: 7 22776 00000 7 |
| Liquid 2 | Grey Splenda ® Zero | UPC Code: 7 22776 00000 7 |
| Liquid 3 | Tate & Lyle Splenda ® | Lot No.: MC22L93141 |
| | Sucralose Concentrate | |

Sucralose-6-acetate was obtained to prepare calibration/validation standards. Salicylic acid was obtained and used as an internal standard. Dextrose and maltodextrin were obtained and used as a blank matrix. All samples were analyzed by UHPLC-MS/MS.

### Methods

### Internal Standard

25.0 mg of salicylic acid was weighed and diluted to 250.0 mL with 1% formic acid in water to produce a concentrated IS solution. 2.0 mL of concentrated IS solution was diluted further to 100.0 mL with 1% formic acid in water to produce an IS solution.

### Blank Matrix

6.0 g of dextrose and 4.0 g of maltodextrin were weighed and diluted with 50.0 mL of IS solution to prepare blank matrix solution.

### Calibration Curve Standards

A 1,000 ppm, or 1.00 mg/mL, standard stock solution was prepared by dissolving 1 mg of sucralose-6-acetate in 1 mL of IS solution. The standard stock solution was diluted further with IS solution as diluent to produce standard working solutions having a concentration range of 8 ng/mL, or 8 ppb, to 700 ng/ml, or 700 ppb.

The aforementioned standard working solutions were diluted 1:1 with blank matrix solution to produce calibration standards having a concentration range of 4 ng/ml, or 4 ppb, to 350 ng/ml, or 350 ppb.

### Quality Control (Validation) Standards

A 1,000 ppm, or 1.00 mg/mL, standard stock solution was prepared by dissolving 1 mg of sucralose-6-acetate in 1 mL of IS solution. The standard stock solution was diluted further with IS solution as diluent to produce standard working solutions having a concentration range of 20 ng/mL, or 20 ppb, to 500 ng/ml, or 500 ppb.

The aforementioned standard working solutions were diluted 1:1 with blank matrix solution to produce validation standards having a concentration range of 10 ng/ml, or 10 ppb, to 250 ng/ml, or 250 ppb.

### Commercial Sucralose-Containing Sample Preparation

1 g of sucralose-containing sample was weighed and diluted with IS solution to generate samples having a concentration of 100 mg/mL.

### Liquid Chromatography

A Shimadzu UHPLC system was used for liquid chromatography, whose components and operating specifications are summarized below in Table 2.

TABLE 2

| Component | Supplier/Specs | |
|---|---|---|
| Pumps | LC-30AD CL | |
| Controller | CBM-20A CL | |
| Column Oven | CTO-20AC CL | |
| Column Temperature | 40° | C. |
| Autosampler | SIL-30AC MP CL | |
| Autosampler Temperature | 4° | C. |
| Degasser | DGU-20A 5R CL | |
| Column | Acquity UPLC BEH C18 | |
| Column Dimensions | 2.1 mm × 100 mm × 1.7 μm | |
| Injection Volume | 10 | μL |
| Flow Rate | 0.2 | mL/min |

The UHPLC mobile phase was a mixture of two mobile phase solvents: Solvent A and Solvent B. Solvent A contained a mixture of 98/2/0.1 (v/v/v)—water, acetonitrile, and formic acid, respectively. Solvent B contained a mixture of 2/98/0.1 (v/v/v)—water, acetonitrile, and formic acid, respectively.

Liquid chromatography was performed using a gradient method described below in Table 3.

TABLE 3

| | |
|---|---|
| Time | Mobile Phase Concentration |

| | | |
|---|---|---|
| 0 | min | 100% Solvent A \| 0% Solvent B |
| 1 | min | 100% Solvent A \| 0% Solvent B |
| 2.5 | min | 80% Solvent A \| 20% Solvent B |
| 11.5 | min | 80% Solvent A \| 20% Solvent B |
| 13 | min | 0% Solvent A \| 100% Solvent B |
| 15.5 | min | 0% Solvent A \| 100% Solvent B |
| 15.51 | min | 100% Solvent A \| 0% Solvent B |
| 18 | min | 100% Solvent A \| 0% Solvent B |

Using the method gradient method above, the retention times of sucralose-6-acetate and salicylic acid were found to be approximately 13.2 min and 12.5 min, respectively.

### Mass Spectrometry

The eluent stream from the UHPLC system during the 0-9 min and 14-18 min time spans above was bypassed from the mass spectrometer detector using a rotary valve bypass. The eluent stream from the UHPLC system during the 9-14 min time span was fed to a Shimadzu 8050 tandem mass spectrometer detector for analysis, the operating specifications of which are summarized below in Table 4.

TABLE 4

| Component | Supplier/Specs |
|---|---|
| Ionization Mode | Multiple Reaction |
| | Monitoring (MRM) Negative |
| | Ion Mode Dual Ion Source |
| | Ionization (DUIS) |
| Ionization Source(s) | Electrospray Ionization (ESI) |
| | Atmospheric Pressure Chemical |
| | Ionization (APCI) |

| | | |
|---|---|---|
| Interface Temperature | 300° | C. |
| Desolvation Line Temperature | 250° | C. |
| Heat Block Temperature | 400° | C. |

| | |
|---|---|
| Drying Gas | Nitrogen |

| | | |
|---|---|---|
| Drying Gas Flow Rate | 10 | L/min |

| | |
|---|---|
| Heating Gas | Nitrogen |

| | | |
|---|---|---|
| Heating Gas Flow Rate | 10 | L/min |

| | |
|---|---|
| Nebulizer Gas | Nitrogen |

| | | |
|---|---|---|
| Nebulizer Gas Flow Rate | 2 | L/min |

| | |
|---|---|
| Collision Gas | Argon |

The mass spectrometer was set to monitor selected ion monitoring (SIM) for sucralose-6-acetate, and multiple reaction monitoring (MRM) for sucralose-6-acetate and salicylic acid (internal standard).

The SIM masses measured for sucralose-6-acetate were m/z=437, 439, 473, 475, 477, 483, 485, 500, 502, 527, 529, 535, 537, 539, 589, 591, 593, 629, 631, 633, 747, 749, 854, 856, 858, 892, and 894.

The MRM transition masses measured for sucralose-6-acetate were m/z=473 to 437, 473 to 439, 483 to 437, and 485 to 439.

The SIM mass measured for salicylic acid was m/z=137.

The MRM transition masses measured for salicylic acid were m/z=137 to 93, 137 to 75, and 137 to 65.

### Quantification

Calibration curve samples, validation samples, and commercial sucralose-containing samples were loaded into autosampler vials or wells of a 96-well plate and maintained at 4° C. prior to injection into the UHPLC system. At the start of the liquid chromatography described above, 50 μL of a single sample was injected into the UHPLC, and the process was repeated until all calibration curve samples, validation samples, and commercial sucralose-containing samples had been injected into the UHPLC system. An exemplary queue for the UHPLC-MS/MS described herein is shown below in Table 5.

TABLE 5

| Sample Type | Concentration | |
|---|---|---|
| Calibration Curve | 4 | ng/mL |
| Calibration Curve | 6 | ng/mL |
| Calibration Curve | 8 | ng/mL |
| Calibration Curve | 10 | ng/mL |
| Calibration Curve | 20 | ng/mL |
| Calibration Curve | 40 | ng/mL |
| Calibration Curve | 60 | ng/mL |
| Calibration Curve | 80 | ng/mL |
| Calibration Curve | 100 | ng/mL |
| Calibration Curve | 150 | ng/mL |
| Calibration Curve | 200 | ng/mL |
| Calibration Curve | 250 | ng/mL |
| Calibration Curve | 300 | ng/mL |
| Calibration Curve | 325 | ng/mL |
| Calibration Curve | 350 | ng/mL |
| IS Solution | Described Above | |
| Matrix Solution | Described Above | |
| Blank | Pure Water | |
| Validation Curve | 10 | ng/mL |
| Validation Curve | 25 | ng/mL |
| Validation Curve | 100 | ng/mL |
| Validation Curve | 250 | ng/mL |
| Blank | Pure Water | |
| Commercial Sucralose-Containing Sample 1 | Described Above | |
| Commercial Sucralose-Containing Sample 2 | Described Above | |
| Commercial Sucralose-Containing Sample 3 | Described Above | |
| Commercial Sucralose-Containing Sample 4 | Described Above | |
| Commercial Sucralose-Containing Sample 5 | Described Above | |
| Commercial Sucralose-Containing Sample 6 | Described Above | |
| Commercial Sucralose-Containing Sample 7 | Described Above | |
| Commercial Sucralose-Containing Sample 8 | Described Above | |
| Commercial Sucralose-Containing Sample 9 | Described Above | |
| Commercial Sucralose-Containing Sample 10 | Described Above | |
| Validation Curve | 10 | ng/mL |
| Validation Curve | 25 | ng/mL |
| Validation Curve | 100 | ng/mL |
| Validation Curve | 250 | ng/mL |
| IS Solution | Described Above | |
| Matrix Solution | Described Above | |
| Blank | Pure Water | |
| Calibration Curve | 4 | ng/mL |
| Calibration Curve | 6 | ng/mL |
| Calibration Curve | 8 | ng/mL |
| Calibration Curve | 10 | ng/mL |
| Calibration Curve | 20 | ng/mL |
| Calibration Curve | 40 | ng/mL |
| Calibration Curve | 60 | ng/mL |
| Calibration Curve | 80 | ng/mL |
| Calibration Curve | 100 | ng/mL |
| Calibration Curve | 150 | ng/mL |
| Calibration Curve | 200 | ng/mL |
| Calibration Curve | 250 | ng/mL |
| Calibration Curve | 300 | ng/mL |
| Calibration Curve | 325 | ng/mL |
| Calibration Curve | 350 | ng/mL |

Sucralose-6-acetate content was individually determined from SIM masses measured and from MRM masses measured.

A SIM-based calibration curve for sucralose-6-acetate were prepared by plotting the SIM area ratio vs. the concentration ratio and fitting this data to a linear best-fit line. The SIM area ratio was obtained by dividing the sucralose-6-acetate peak area determined from the total ion count (“TIC”) of all masses measured in SIM by the salicylic acid peak area determined from the TIC of all masses measured in SIM. The concentration ratio was obtained by dividing the sucralose-6-acetate concentration corresponding to a given peak area by the salicylic acid concentration corresponding to a given peak area.

An MRM-based calibration curve for sucralose-6-acetate were prepared by plotting the MRM area ratio vs. the concentration ratio and fitting this data to a linear best-fit line. The MRM area ratio was obtained by dividing the sucralose-6-acetate peak area determined from the TIC of all masses measured in MRM by the salicylic acid peak area determined from the TIC of all masses measured in MRM. The concentration ratio was obtained by dividing the sucralose-6-acetate concentration corresponding to a given peak area by the salicylic acid concentration corresponding to a given peak area.

Concentrations of sucralose-6-acetate in the validation samples and commercial sucralose-containing samples were determined by calculating their concentration from their measured peak area using the calibration curves and was determined for both the SIM and MRM calibration curves.

The lower limit of quantitation (LLOQ) and upper limit of quantitation (ULOQ) for the calibration curve were determined when: (i) the linear best-fit line had an R² value of 0.985; and (ii) at least 75% and a minimum of six non-zero calibration samples were within 15% of their nominal concentration (for samples not within 20% of the LLOQ).

The acceptance criteria for this analytical method was set such that: (i) at least 67% of validation samples needed to be within 15% of their nominal value; and (ii) 50% of validation samples per level, e.g., O (1 ng/mL), O (10 ng/mL), etc., needed to be within 15% of their nominal value.

The limit of detection was determined by multiplying the standard deviation of the LLOQ by 3 (for n=5 samples).

Any sample for which the calculated concentration of sucralose-6-acetate was below the LLOQ was reported as “<LOQ”. Any sample for which the calculated concentration of sucralose-6-acetate was above the ULOQ was prepared again at a more dilute concentration, i.e., less than 100 mg/mL, and reanalyzed until the calculated concentration of sucralose-6-acetate was below ULOQ.

After all acceptance criteria were satisfied and sucralose-6-acetate concentration in the commercial sucralose-containing samples were determined, the concentration was converted to a mass percentage (in ng/g or ppb) by Equation 1 above.

### Results

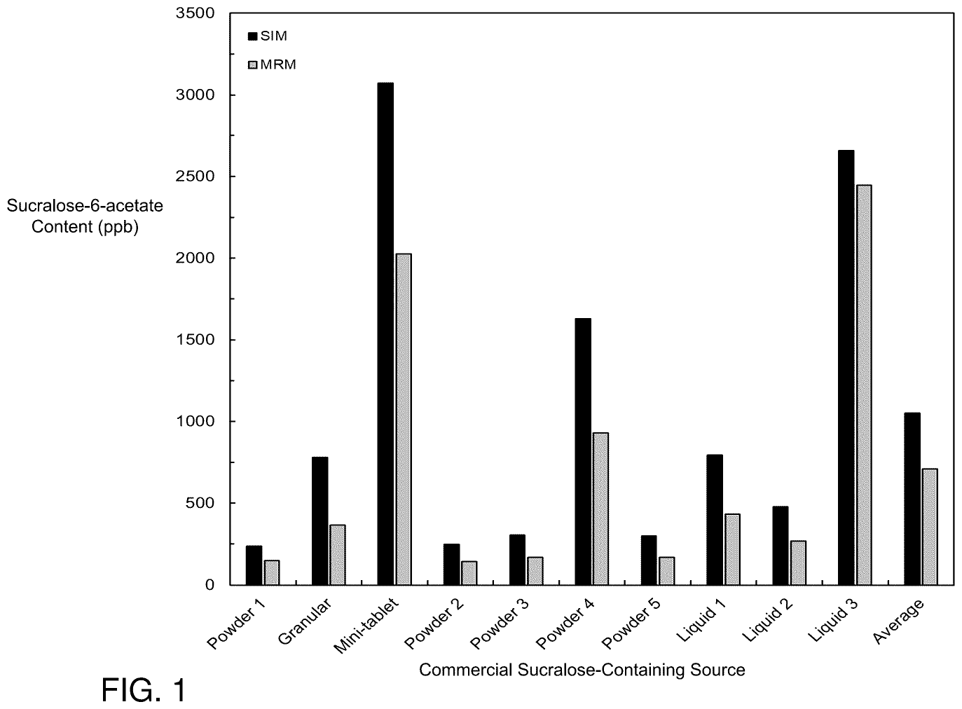

All ten commercial sucralose-containing samples contained sucralose-6-acetate, as shown in FIG. 1. The lower limit of quantitation for the method of Example 1 was found to be 1 ppb.

When analyzed using SIM, the sucralose-6-acetate content of the ten commercial sucralose-containing samples ranged from about 237 ppb to about 3073 ppb, or 235-3073 ng sucralose-6-acetate per g of sample, with an average of about 1049 ppb, or 1049 ng sucralose-6-acetate per g of sample. The full results for analysis using SIM are reported below in Table 6. Notably, eight of the ten commercial sucralose-containing samples analyzed (80%) contained an amount of sucralose-6-acetate per serving that would exceed the potentially genotoxic limit, if one serving of the commercial product were consumed daily.

TABLE 6

| Commercial | Sucralose-6- | | Amount of | | Sucralose-6- | | Exceeds 0.15 μg |
|---|---|---|---|---|---|---|---|
| Sucralose- | acetate | | Product per | | acetate Content | | Sucralose-6-acetate |
| Containing Sample | Content¹ | | Serving | | per serving | | per Serving? |
| Powder 1 | 237 | ng/g | 1000 | mg | 0.235 | μg | Yes |
| Granular Solid 1 | 776 | ng/g | 1000 | mg | 0.781 | μg | Yes |
| Mini-tablet | 3064 | ng/g | 14 | mg | 0.043 | μg | No |
| Powder 2 | 242 | ng/g | 1000 | mg | 0.248 | μg | Yes |
| Powder 3 | 300 | ng/g | 1000 | mg | 0.304 | μg | Yes |
| Powder 4 | 1624 | ng/g | 1000 | mg | 1.629 | μg | Yes |
| Powder 5 | 290 | ng/g | 1000 | mg | 0.297 | μg | Yes |
| Liquid 1² | 789 | ng/g | 500 | mg | 0.396 | μg | Yes |
| Liquid 2² | 467 | ng/g | 500 | mg | 0.239 | μg | Yes |
| Liquid 3³ | 2650 | ng/g | 40 | mg | 0.106 | μg | No |

¹Units are ng of sucralose-6-acetate per g of product.
²Liquids 1 and 2 had a serving size of 0.5 mL, which corresponds to 0.5 g, or 500 mg.
³Liquid 3 had a serving size of 0.04 mL, which corresponds to 0.04 g, or 40 mg.

When analyzed using MRM, the sucralose-6-acetate content of the ten commercial sucralose-containing samples ranged from about 143 ppb to about 2447 ppb, 0.143-2.447 ppm, with an average of about 709 ppb, or 0.709 ppm. The full results for analysis using MRM are reported below in Table 7. Notably, five of the ten commercial sucralose-containing samples analyzed (50%) contained an amount of sucralose-6-acetate per serving that would exceed the potentially genotoxic limit, if one serving of the commercial product were consumed daily.

TABLE 7

| Commercial | Sucralose-6- | | Amount of | | Sucralose-6- | | Exceeds 0.15 μg |
|---|---|---|---|---|---|---|---|
| Sucralose- | acetate | | Product per | | acetate Content | | Sucralose-6-acetate |
| Containing Sample | Content¹ | | Serving | | per serving | | per Serving? |
| Powder 1 | 144 | ng/g | 1000 | mg | 0.144 | μg | No |
| Granular Solid 1 | 365 | ng/g | 1000 | mg | 0.365 | μg | Yes |
| Mini-tablet | 2016 | ng/g | 14 | mg | 0.028 | μg | No |
| Powder 2 | 136 | ng/g | 1000 | mg | 0.136 | μg | No |
| Powder 3 | 158 | ng/g | 1000 | mg | 0.158 | μg | Yes |
| Powder 4 | 924 | ng/g | 1000 | mg | 0.924 | μg | Yes |
| Powder 5 | 164 | ng/g | 1000 | mg | 0.164 | μg | Yes |
| Liquid 1² | 427 | ng/g | 500 | mg | 0.214 | μg | Yes |
| Liquid 2² | 259 | ng/g | 500 | mg | 0.130 | μg | No |
| Liquid 3³ | 2440 | ng/g | 40 | mg | 0.098 | μg | No |

¹Units are ng of sucralose-6-acetate per g of product.
²Liquids 1 and 2 had a serving size of 0.5 mL, which corresponds to 0.5 g, or 500 mg.
³Liquid 3 had a serving size of 0.04 mL, which corresponds to 0.04 g, or 40 mg.

Collectively, the results presented herein demonstrate that at least 50% of commercially available sucralose-containing samples analyzed contained an amount of sucralose-6-acetate that is potentially genotoxic, if a single serving of that product were consumed per day, regardless of whether SIM or MRM analysis is used. Since all of the sucralose-containing samples analyzed herein were obtained via commercial sources, these results highlight that the existing analytical methods in this field for quantifying sucralose-6-acetate content clearly lack the ability to do so at the relevant levels to ensure that a potentially genotoxic level of sucralose-6-acetate is not present in sucralose, or the products that contain it. As such, the analytical methods described herein are an essential tool that can, and should, be used to ensure consumers that sucralose-containing food products are safe for consumption.

### Example 2

Commercially available sucralose-containing beverages, nutritional drinks, supplements, energy drinks, colas, flavored waters, flavored syrups, and the like, will be obtained.

These samples will be prepared and analyzed for sucralose-6-acetate content using methods consistent with those detailed in Example 1.

Regarding results, the analysis will demonstrate that the samples will contain sucralose-6-acetate. The analysis will also demonstrate that the content of some samples will exceed 0.15 μg per serving. The analysis will further demonstrate a lower limit of quantitation for the method of about 1 ppb.

### Example 3

Commercially available sucralose-containing beverages, nutritional drinks, supplements, energy drinks, colas, flavored waters, flavored syrups, and the like, will be obtained.

These samples will be prepared and analyzed for sucralose-6-acetate content using methods consistent with those detailed in Example 1, with the differences noted below.

The concentrations of the sucralose-containing samples, calibration curve samples, and validation standards will be prepared at concentrations as described herein.

The LC method will use a gradient method, solvents, and run time as described herein.

Regarding results, the analysis will demonstrate that the samples will contain sucralose-6-acetate. The analysis will also demonstrate that the content of some samples will exceed 0.15 μg per serving. The analysis will further demonstrate a lower limit of quantitation for the method of about 1 ppb.

### Example 4

Commercially available sucralose-containing powders, liquids, granular solids, tablets, beverages, nutritional drinks, supplements, energy drinks, colas, flavored waters, flavored syrups, and the like, will be obtained.

The sucralose-containing samples, calibration curve samples, and validation standards prepared and analyzed for sucralose-6-acetate content using methods consistent with those detailed in Example 1, with the differences noted below.

The blank matrix will be water rather than 60% dextrose/40% maltodextrin.

The concentrations of the sucralose-containing samples, calibration curve samples, and validation standards will be prepared at concentrations as described herein.

The LC method will use a gradient method, solvents, and run time as described herein.

Regarding results, the analysis will demonstrate that the samples will contain sucralose-6-acetate. The analysis will also demonstrate that the content of some samples will exceed 0.15 μg per serving. The analysis will further demonstrate a lower limit of quantitation for the method of about 1 ppb.

### Example 5

Commercially available sucralose-containing powders, liquids, granular solids, tablets, beverages, nutritional drinks, supplements, energy drinks, colas, flavored waters, flavored syrups, and the like, will be obtained.

The sucralose-containing samples, calibration curve samples, and validation standards prepared and analyzed for sucralose-6-acetate content using methods consistent with those detailed in Example 4, with the differences noted below.

The blank matrix will contain sugar.

The concentrations of the sucralose-containing samples, calibration curve samples, and validation standards will be prepared at a concentrations as described herein.

The LC method will use a gradient method, solvents, and run time as described herein.

Regarding results, the analysis will demonstrate that the samples will contain sucralose-6-acetate. The analysis will also demonstrate that the content of some samples will exceed 0.15 μg per serving. The analysis will further demonstrate a lower limit of quantitation for the method of about 1 ppb.

The present disclosure is not to be limited in terms of the particular embodiments described in this application, which are intended as illustrations of various aspects of the present disclosure. Many modifications and variations can be made without departing from its spirit and scope. Functionally equivalent methods and apparatuses within the scope of the disclosure, in addition to those enumerated herein, are possible from the foregoing descriptions. Such modifications and variations are intended to fall within the scope of the appended claims. The terminology used herein is for the purpose of describing particular embodiments only and is not intended to be limiting unless otherwise set forth herein.

1.-154. (canceled)

155. A method of determining an amount of sucralose-6-acetate in a sucralose-containing material, the method comprising:
(i) preparing one or more calibration samples, wherein each of the one or more calibration samples comprise a concentration of sucralose-6-acetate;
(ii) preparing a sucralose-containing material sample, wherein the sucralose-containing material sample comprises the sucralose-containing material;
(iii) providing a liquid chromatography separation unit comprising:
a chromatographic column; and
a mobile phase;
(iv) providing a mass spectrometer downstream of the liquid chromatography separation unit, wherein the mass spectrometer is configured to:
receive an eluent of the liquid chromatography separation unit;
determine a total ion count of the eluent for at least one mass-to-charge ratio that is characteristic of sucralose-6-acetate; and
generate a chromatogram based on the total ion count, wherein the chromatogram comprises a peak associated with sucralose-6-acetate, and wherein the peak associated with sucralose-6-acetate comprises a peak area;
(v) providing a liquid chromatography method configured to be implemented on the liquid chromatography separation unit when a sample is injected into the liquid chromatography separation unit, wherein the liquid chromatography method specifies:
a run time;
a composition of the mobile phase during the run time; and
a flow rate of the mobile phase during the run time;
(vi) injecting a single sample selected from the one or more calibration samples and the sucralose-containing material sample into the liquid chromatography separation unit and implementing the liquid chromatography method;
(vii) repeating step (vi) until all of the one or more calibration samples and the sucralose-containing material sample have been injected into the liquid chromatography separation unit;
(viii) generating a standard curve by:
for each of the one or more calibration samples, plotting the peak area against the concentration of sucralose-6-acetate on a plot; and
fitting the plot with a best-fit line; and
(ix) determining the amount of sucralose-6-acetate in the sucralose-containing material from the standard curve using the peak area of the sucralose-containing material sample.

156. The method of claim 155, wherein the sucralose-containing material is in a form selected from the group consisting of a powder, a granular solid, a crystalline solid, and a liquid.

157. The method of claim 155, wherein the sucralose-containing material comprises at least one selected from the group consisting of dextrin, dextrose, maltodextrin, and any combination thereof.

158. The method of claim 155, wherein the composition of the mobile phase is constant during the run time.

159. The method of claim 155, wherein the composition of the mobile phase is different at least two time points during the run time.

160. The method of claim 155, wherein the amount of sucralose-6-acetate in the sucralose-containing material is between about 1 ppb and about 5,000 ppm relative to sucralose.

161. The method of claim 160, wherein the amount of sucralose-6-acetate in the sucralose-containing material is between about 1 ppb and about 10 ppm relative to sucralose.

162. The method of claim 161, wherein the amount of sucralose-6-acetate in the sucralose-containing material is between about 1 ppb and about 1 ppm relative to sucralose.

163. The method of claim 162, wherein the chromatographic column is selected from the group consisting of a C30 column, a C18 column, a C8 column, a C4 column, a polar-endcapped C18 column, an amide-embedded C18 column, a sulfonamide embedded C18 column, a phenyl column, a phenyl-hexyl column, a biphenyl column, a pentafluorophenyl column, and a hydrophilic interaction column.

164. The method of claim 163, wherein the chromatographic column is a C18 column.

165. The method of claim 163, wherein the chromatographic column is a hydrophilic interaction column.

166. The method of claim 155, wherein the at least one mass-to-charge ratio that is characteristic of sucralose-6-acetate is selected from the group consisting of 437, 439, 473, 475, 477, 483, 485, 500, 502, 527, 529, 535, 537, 539, 589, 591, 593, 629, 631, 633, 747, 749, 854, 856, 858, 892, 894, and any combination thereof.

167. The method of claim 155, wherein the run time is at least 5 minutes.

168. The method of claim 155, wherein the run time is at least 10 minutes.

169. The method of claim 155, wherein the method comprises a separation factor of at least 2 minutes.

170. The method of claim 155, wherein the sucralose-containing material is a solid that comprises maltodextrin and dextrose.

171. The method of claim 155, wherein the mobile phase comprises a mixture of a first liquid solvent (Solvent A) comprising about 98% water, about 2% organic solvent, and about 0.1% acid additive (by volume), and a second liquid solvent (Solvent B) comprising about 2% water, about 98% organic solvent, and about 0.1% acid additive (by volume).

172. The method of claim 171, wherein the organic solvent is acetonitrile.

173. The method of claim 171, wherein the run time is about 18 minutes and wherein the composition of the mobile phase is:
(i) about 100% Solvent A and about 0% Solvent B at the start of the run time;
(ii) about 100% Solvent A and about 0% Solvent B at about 1 minute into the run time;
(iii) about 80% Solvent A and about 20% Solvent B at about 2.5 minutes into the run time;
(iv) about 80% Solvent A and about 20% Solvent B at about 11.5 minutes into the run time;
(v) about 0% Solvent A and about 100% Solvent B at about 13 minutes into the run time;
(vi) about 0% Solvent A and about 100% Solvent B at about 15.5 minutes into the run time;
(vii) about 100% Solvent A and about 0% Solvent B at about 15.51 minutes into the run time; and
(viii) about 100% Solvent A and about 0% Solvent B at about 18 minutes into the run time.

174. The method of claim 173, the eluent is diverted from the mass spectrometer until about 9 minutes into the run time.
